(19) United States
(12) Patent Application Publication
(10) Pub. No.: US 2002/0048802 A1
(43) Pub. Date: Apr. 25, 2002

(54) PROKARYOTIC REVERSE TRANSCRIPTASE

(76) Inventors: SUMIKO INOUYE, BRIDGEWATER, NJ (US); MEI-YIN HSU, HILLSBOROUGH, NJ (US); MASAYORI INOUYE, BRIDGEWATER, NJ (US); SUSAN EAGLE, BRIDGEWATER, NJ (US); BERT C. LAMPSON, KNOXVILLE, TN (US); JUNG SUN, WOODBRIDGE, NJ (US); JORGE VALLEJO-RAMIREZ, NEWARK, NJ (US)

Correspondence Address: SCHNADER HARRISON SEGAL & LEWIS, LLP, 1600 MARKET STREET, SUITE 3600, PHILADELPHIA, PA, 19103

(21) Appl. No.: 08/808,031

(22) Filed: Mar. 3, 1997

Related U.S. Application Data
(63) Continuation of application No. 08/269,118, filed on Jun. 30, 1994, now abandoned.
Application No. 08/269,118 is a continuation-in-part of application No. 07/315,427, filed on Feb. 24, 1989.
Application No. 07/315,427 is a continuation-in-part of application No. 07/315,316, filed on Feb. 24, 1989.
Application No. 07/315,316 is a continuation-in-part of application No. 07/315,432, filed on Feb. 24, 1989, now abandoned.
Application No. 07/315,432 is a continuation-in-part of application No. 07/517,946, filed on May 2, 1990, now abandoned.
Application No. 07/517,946 is a continuation-in-part of application No. 07/518,749, filed on Mar. 2, 1990.
Application No. 07/518,749 is a continuation-in-part of application No. 07/753,110, filed on Aug. 30, 1991.
Application No. 07/753,110 is a continuation-in-part of application No. 07/817,430, filed on Jan. 6, 1992.
Application No. 07/817,430 is a continuation-in-part of application No. 07/979,447, filed on Nov. 20, 1992, now abandoned.

(51) Int. Cl. C12N 9/12

(52) U.S. Cl. 435/194

(57) ABSTRACT

The present invention relates to a prokaryotic reverse transcriptase enzyme. The enzyme is capable of synthesizing a hybrid DNA-RNA molecule called msDNA with the genes which synthesize the DNA and RNA portions of the molecule.

### RELATED CASES

This is a continuation-in-part of prior copending U.S. patent application Ser. No. 07/315,427, filed Feb. 24, 1989 and since issued as U.S. Pat. No. 5,079,151 on Jan. 7, 1992, which is a continuation-in-part of prior copending U.S. patent application Ser. No. 07/315,316, filed Feb. 24, 1989 and since issued as U.S. Pat. No. 5,320,958 on Jun. 14, 1994, which is a continuation-in-part of prior copending U.S. patent application Ser. No. 07/315,432, filed on Feb. 24, 1989 and since abandoned, which is a continuation -in-part of prior copending U.S. patent application Ser. No. 07/517,946, filed May 2, 1990, which is a continuation-in-part of prior copending, U.S. patent application Ser. No. 07/518,749, filed on Mar. 2, 1990, which is a continuation-in-part of prior copending U.S. patent application Ser. No. 07/753,110, filed on Aug. 30, 1991, which is a continuation-in-part of prior copending U.S. patent application Ser. No. 07/817,430, filed Jan. 6, 1992, which is a continuation-in-part of prior U.S. patent application Ser. No. 07/979,447, filed Nov. 20, 1992, respectively which are incorporated herein by reference.

### FIELD OF THE INVENTION

The invention relates to bacterial RT enzymes which are capable of synthesizing a hybrid RNA-DNA molecule called msDNA together with the genes which synthesize the DNA and RNA portion of the molecule.

Another aspect of the invention relates to the isolation and purification of RTs from bacterium which is capable of synthesizing msDNA. The invention deals with groups of prokaryotes e.g., bacteria which are capable of synthesizing msDNAs by means of a reverse transcriptase. The bacterium capable of synthesizing msDNAs is identified by testing positive by an appropriate screening test.

This is the first time that, as taught in the subject parent patent applications, reverse transcriptase has been found and isolated from a prokaryote.

### BACKGROUND OF THE INVENTION

Previously, there was described a chromosomal region of the bacterium Myxococcus xanthus which coded for the RNA and DNA portions of an msDNA. Dhundale et al. (Dhundale '87) “Structure of msDNA from Myxococcus xanthus: Evidence for a Long, Self-Annealing RNA precursor for the Covalently Linked, Branched RNA”, Cell, Vol. 51, pages 1105-1112 (Dec. 24, 1987). Dhundale et al. speculated that an AluI nucleotide fragment contained all the essential coding regions to produce an msDNA. This speculation turned out to be in error.

This AluI fragment of Dhundale et al., in fact, and inherently did not contain the gene sequence coding for an RT. The AluI fragment was too short to code for the gene sequence coding for and RT. This was proven by way of sequence analysis by a computer program which searches for open reading frames that can potentially code for a protein. The print-out of the sequence analysis clearly shows that there is no translational reading frame in the Dhundale et al. fragment open across a stretch of DNA sufficiently long enough to encode any reverse transcriptase.

What is reported in Dhundale et al. in 1987 with respect to a bacterial reverse transcriptase was totally contrary to accepted dogma at that time about the distribution of these enzymes, i.e., that they were present only in viruses which infect eukaryotic organisms.

For the 20 years since the discovery of reverse transcriptase, it was believed that these enzymes were restricted to viruses which infect eukaryotic cells. Now, in accordance with the invention, reverse transcriptases have been identified in bacteria.

### SUMMARY OF THE INVENTION

In accordance with the invention, it is shown that various bacteria have nucleotide sequences named “retrons” which encode reverse transcriptase (RTs) which are capable of synthesizing msDNAs. The invention also relates to the isolated and purified bacterial RTs. It has also been determined that the RTs of the bacteria which synthesize msDNAs possess common conserved nucleotide sequences and amino acid residues.

Representative members of the Enterobacteriaceae, Rhizobiaceae and Mycobacteriaceae families are demonstrated to be capable of synthesizing msDNA. These bacteria can be screened for the capability of synthesizing msDNA by an RT labeling or extension in vitro test.

### BRIEF DESCRIPTION OF THE DRAWINGS

FIG. 1 shows the restriction map of the 3.4 kb fragment around msd and downstream of msr.

FIG. 2 shows the nucleotide sequence of the chromosomal region encompassing the msDNA and msdRNA coding regions and an ORF region downstream of msr and the amino acid sequence of Mx162-RT.

FIG. 3 shows the amino acid sequence alignment of the msDNA-Mx162 ORF with a portion of the retroviral Pol sequence from HIV and HTLV1 and the ORF of msDNA-Ec67.

FIG. 4 shows the sequence similarity of the msDNA-Mx162 reverse transcriptase with other retroelements.

FIG. 5 shows the sequence comparison of the regions around the YXDD box of various reverse transcriptases.

FIG. 6 shows the detection of the msDNA in a clinical isolate of E. coli.

FIG. 7 shows the complete primary and proposed secondary structure of msDNA-Ec67.

FIG. 8 shows the determination of the RNA nucleotide sequence for the branched RNA linked to msDNA.

FIG. 9 shows the southern blot analysis of E. coli Cl-1 Chromosomal DNA(A) and analysis of msDNA synthesis by pCl-1E and pCl-1P(B).

FIG. 10 shows the restriction map of the 11.6 kb Eco RI fragment.

FIG. 11 shows the nucleotide sequence of the region from the E. coli Cl-1 chromosome encompassing the msDNA, msdRNA and ORF coding regions and the amino acid sequence of Ec67-RT.

FIG. 12 shows the amino acid sequence alignment of the E. coli msDNA ORF with a portion of the retroviral Pol sequence from HIV and HTLV1.

FIG. 13 shows the detection of RT activity from various cell extracts.

FIG. 14 shows the amino acid sequence alignment of bacterial RTs.

FIG. 15 shows the nucleotide and amino acid sequence of Mx65-RT.

FIG. 16 shows the nucleotide and amino acid sequence of Sa163-RT.

FIG. 17 shows the nucleotide and amino acid sequence of Ec73-RT.

FIG. 18 shows the nucleotide and amino acid sequence of Ec86-RT.

FIG. 19 shows the nucleotide and amino acid sequence of Ec107-RT.

FIG. 20 shows the msDNAs from total RNA prepared from each bacterial strain were specifically labeled with ³²P by the RT extension method (12, 14).

FIG. 21 shows a collection of 63 rhizobial isolates screened for the presence of msDNA by the RT extension method.

### DETAILED DESCRIPTION OF THE DRAWINGS

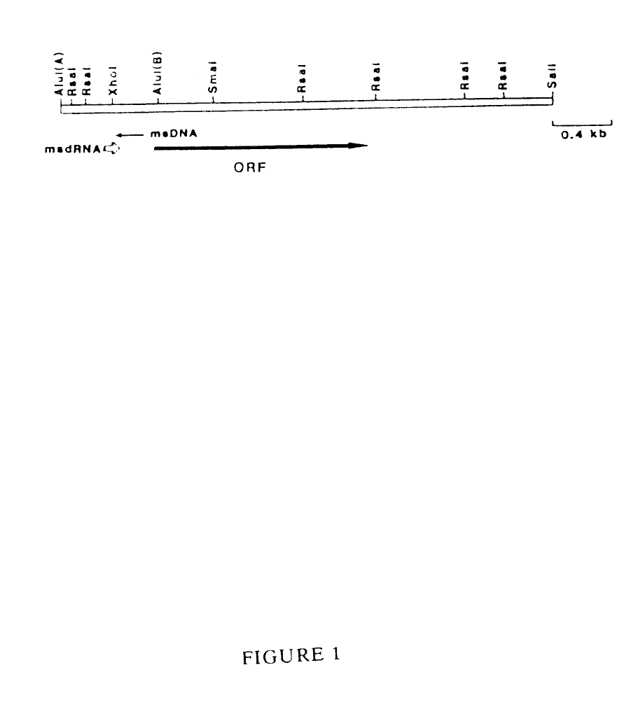

FIG. 1. Restriction Map of the 3,4-kb fragment Around msd and Downstream of msr.

The locations and the orientation of msDNA and msdRNA are indicated by a small arrow and an open arrow, respectively. A large solid arrow represents an ORF and its orientation. The only two AluI sites (A and B) are shown and the DNA sequence between AluI (A) and AluI (B) was determined previously by Yee et al. (1984).

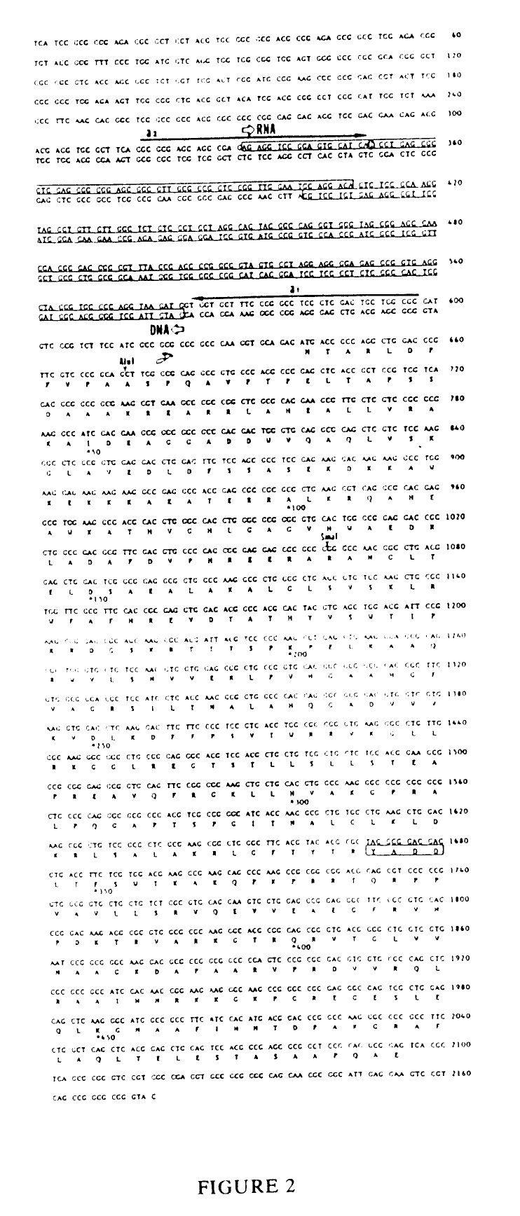

FIG. 2. Nucleotide Sequence of the Chromosomal Region Encompassing the msDNA and msdRNA Coding Regions and an ORF Region Downstream of msr.

The upper strand beginning at the AluI (A) site (see FIG. 1) and ending just beyond the ORF is shown. Only a part of the complementary lower strand is shown from base-301 to-600. The boxed region of the upper strand (332-408) and the boxed region of the lower strand (401-562) correspond to the sequences of msdRNA and msDNA respectively (Dhundale et al., 1987). The starting sites for DNA and RNA and the 5′ to 3′ orientations are indicated by open arrows. The msdRNA and msDNA regions overlap at their 3′ ends by 8 bases. The circled G residue at position 351 represents the branched rG of RNA linked to the 5′ end of the DNA strand in msDNA. Long solid arrows labeled a1 and a2 represent inverted repeat sequences proposed to be important in the secondary structure of the primary RNA transcript involved in the synthesis of msDNA (Dhundale et al., 1987). The ORF begins with the initiation codon at base 640. Single letter designations are given for amino acids. The YXDD amino acid sequence highly conserved among known RT proteins is boxed. Numbers on the right hand column enumerate the nucleotide bases and numbers with a* enumerate amino acids. Small vertical arrows labeled AluI and SmaI locate the AluI and SmaI restriction cleavage sites, respectively. The DNA sequence was determined by the chain termination method (Sanger et al., 1977) using synthetic oligonucleotides as primer.

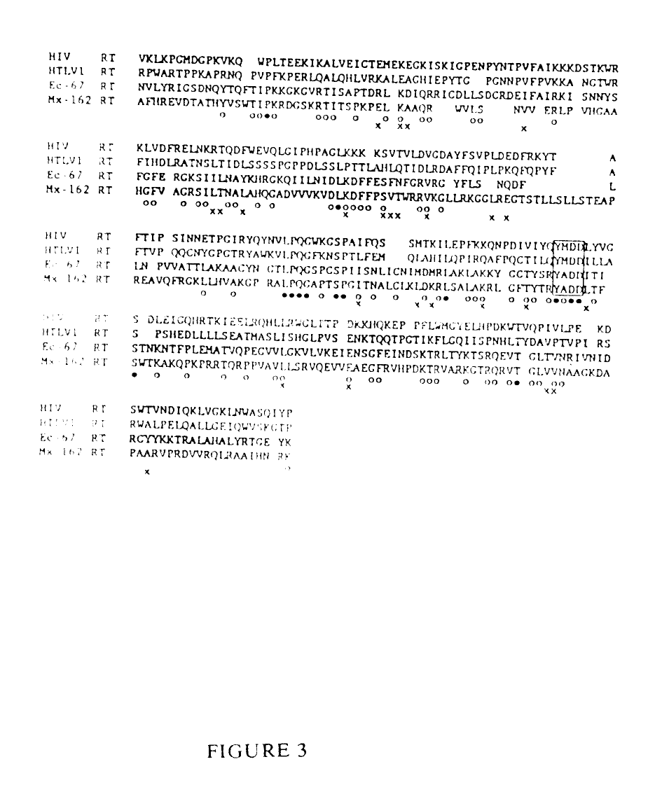

FIG. 3. Amino acid Sequence Alignment of the msDNA-Mx162/ORF with a Portion of the Retroviral Pol Sequences from HIV and HTLV1 and the ORF of msDNA-Ec67.

Amino acid sequences are compared with matching residues assigned as follows: (o) amino acid residues shared by all four proteins; (o) amino acid residues shared by msDNA-Mx162 and msDNA-Ec67 RTs; (x) amino acid residues shared by msDNA-Mx162 RT with HIV or HTLV1 RTs. Amino acid sequences showed are from residue-177 to-439 for HIV RT (Ratner et al., 1985); residue-15 to-277 for HTLV1 RT (Seiki et al., 1983); residue-32 to-291 for Ec-67 RT (Lampson et al., 1989); and residue-170 to-435 for Mx-162 RT (this work). The YXDD consensus sequence is outlined in a box.

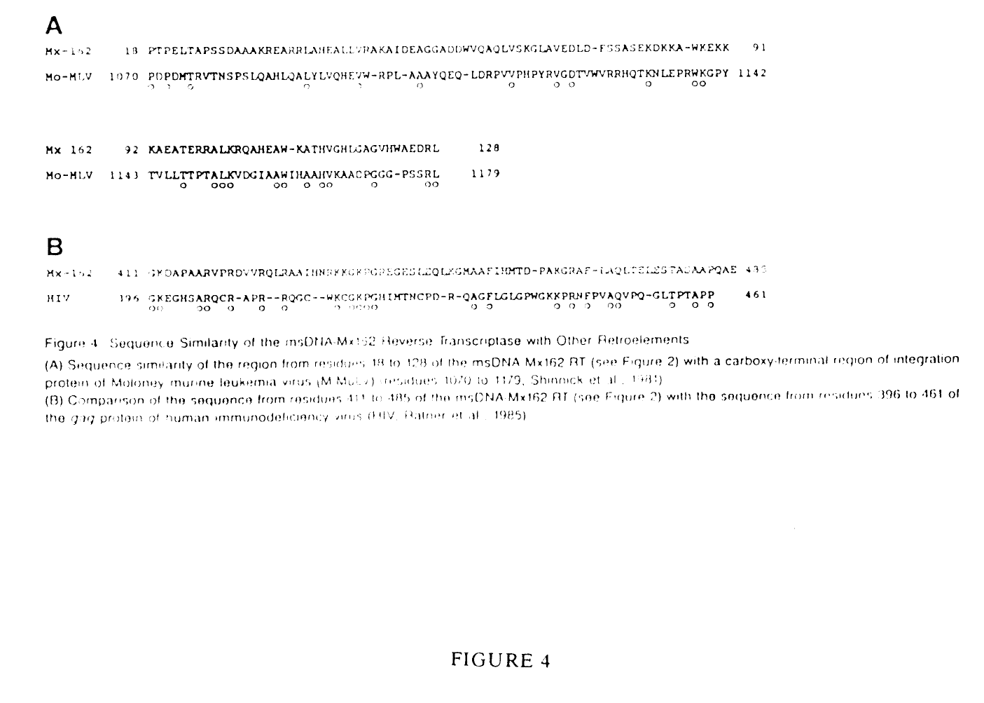

FIG. 4. Sequence Similarity of the msDNA-Mx162 Reverse Transcriptase with Other Retroelements. A. Sequence similarity of the region from residue-18 to-128 of the msDNA-Mx162 RT (see FIG. 2) with a carboxyl terminal region of integrase of Moloney murine leukemia virus (Mo-MLV) (residue-1070 to-1179; Shinnick et al., 1981). B. Comparison of the sequence from residue-411 to-485 of the msDNA-Mx162 RT (see FIG. 2) with the sequence from residue-396 to-461 of the gap protein of human immunodeficiency virus (HIV; Ratner et al., 1985).

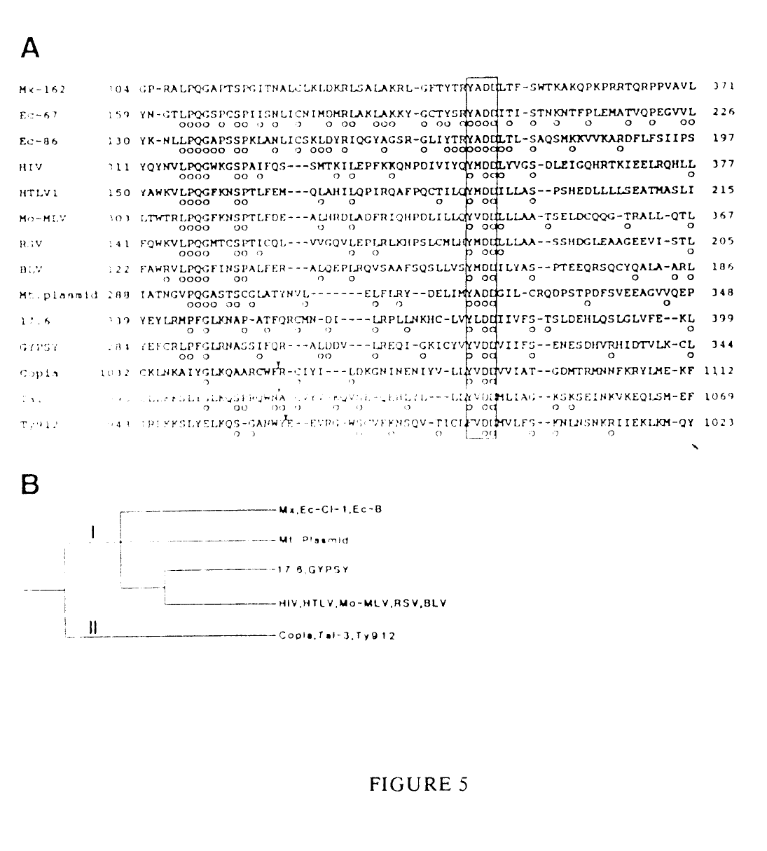

FIG. 5. Sequence Comparison of the Regions Around the YXDD Box of Various Reverse Transcriptases.

The region from residue-304 to residue-371 of the msDNA-Mx162 RT (see FIG. 2) is aligned with various RTs from different sources. The identical amino acid residues with the msDNA-Mx162 RT are indicated by open circles. The YXDD sequences are boxed. The residue numbers for the amino terminal residues and for the carboxyl terminal residues are indicated by the left and the right hand sides of the sequences, respectively. Mx-162 RT from this work (FIG. 2); Ec-67 RT from Lampson et al. (1989); Ec-86 RT from Lim and Maas (1989); HIV RT from Ratner et al. (1985); HTLV1 RT from Seiki et al. (1983); Mo-MLV RT from Shinnick et al. (1981); RSV (Rous sarcoma virus) RT from Dickson et al. (1982); BLV (bovine leukemia virus) RT from Rice et al. (1985): Mt. plasmid (Neurosporo mitochondrial plasmid) RT from Nargang et al. (1984); 17.6 Drosophila retrotransposon from Saigo et al. (1984); gypsy Drosophila retrotransposon from Yuki et al. (1986); Tal-3 plant (Arabidopsis thaliana) retransposon from Voytas and Ausubel (1988); and Ty912 yeast retrotransposon from Clare and Farabaugh (1985). Small arrows in Copia, Tal-3 and Ty912 indicate positions of insertions of extra sequences of 18, 18 and 13 residues, respectively. B. Phylogenetic relationships among various RTs listed in A. The branching positions are arbitrarily illustrated.

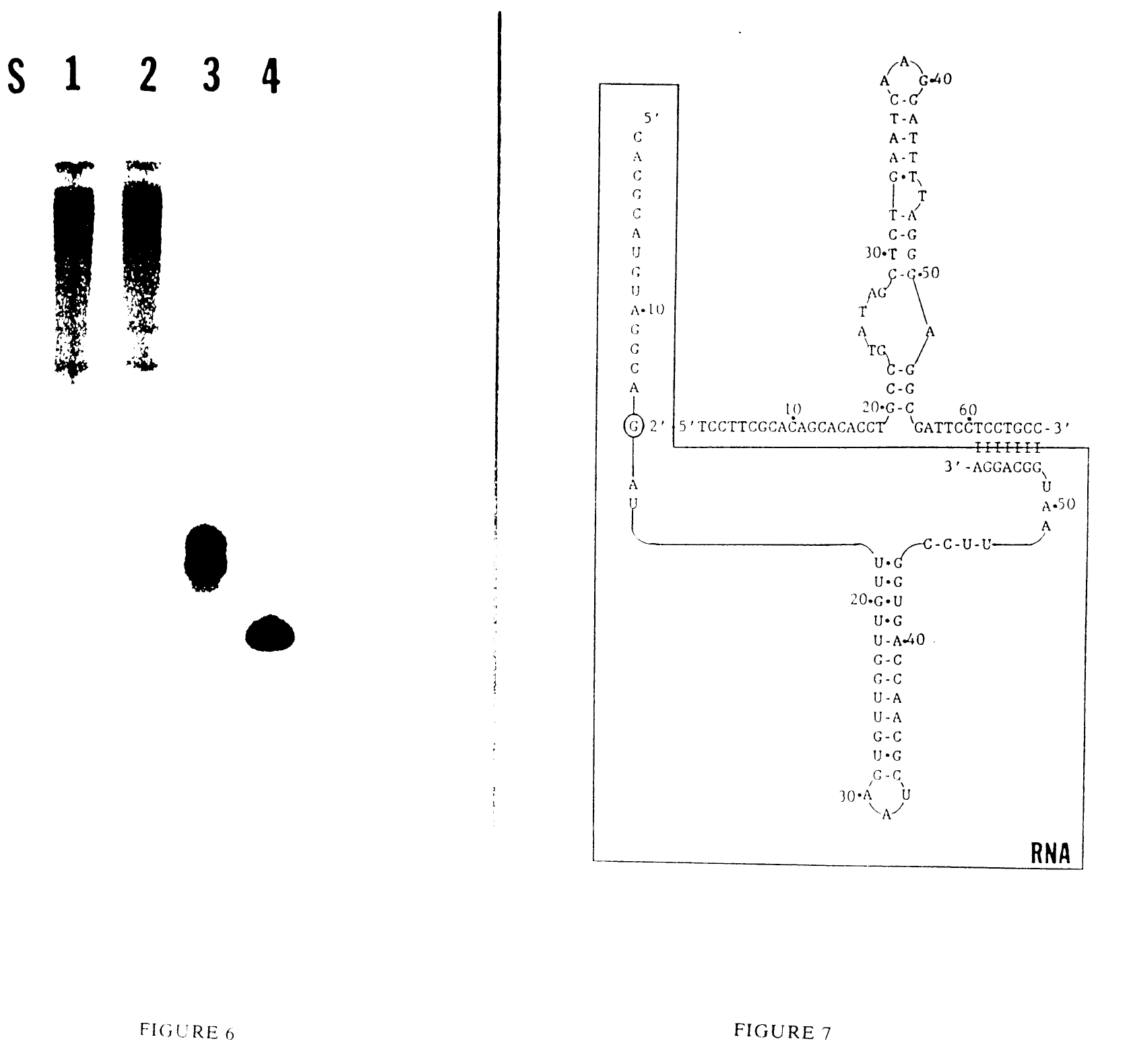

FIG. 6. Detection of msDNA in a clinical isolate of E. coli. Total RNA, prepared (Maniatis et al., 1982) from a 5-ml culture, was added to 50 μl of a reaction mixture containing: 50 mM tris-HCl (pH8.3); 6 mM MgCl₂; 40 mM KCl; 5 mM DTT; 1 μM dATP, dTTP, and dGTP; 0.04 μM dCTP; 0.2 μM |α-³²P|dCTP; and 10 units of AMV-RT (Bochringer Mannheim). The reaction mixture was incubated at 37° C. for 30 min. followed by extraction with 50 μl phenolchloroform (1:1) and ethanol precipitation. The samples were electrophoresed on a 4% acrylamide-8 M urea gel. Lanes (S) molecular weight markers; MspI digest of pBR322 end-labeled with |α-³²P|dCTP and the Klenow fragment of DNA polymerase I. (1) E. coli K-12 strain (600, (2) the same as in lane 1 except the sample was treated with RNase A (5 μg, 10 min at 37° C.) just prior to electrophoresis. (3) clinical isolate Cl-1. (4 ) clinical isolate Cl-1 treated with RNase A. The clinical isolate was identified as Escherichia coli (The clinical E. coli strains were urinary tract isolates kindly provided by Dr. Melvin Weinstein from the microbiology laboratory. R. W. Johnson Hospital, New Brunswick, N.J. The clinical strain Cl-1 was identified using the API-20E identification system (API laboratory products) and gave a typical E. coli profile number of 5044552).

FIG. 7. The complete primary and proposed secondary structure of msDNA-Ec67. The DNA sequence was determined by the Maxam and Gilbert method (Maxam et al., 1980) using 3′-end labeled msDNA. The RNA sequence (msdRNA; boxed region) was determined using base-specific RNases as previously described (Dhundale et al., 1987). The 2′,5′ Branched linkage between the 15th rG residue and the 5′ end of the DNA strand was determined using the debranching enzyme from HeLa cells as described previously (Dhundale et al., 1987; Furuichi et al., 1987; Ruskin et al., 1985; Arenas et al., 1987; the debranching enzyme was a gift from Jerard Hurwitz). The branched rG at position 15 is circled, and both RNA and DNA are numbered from their 5′ ends.

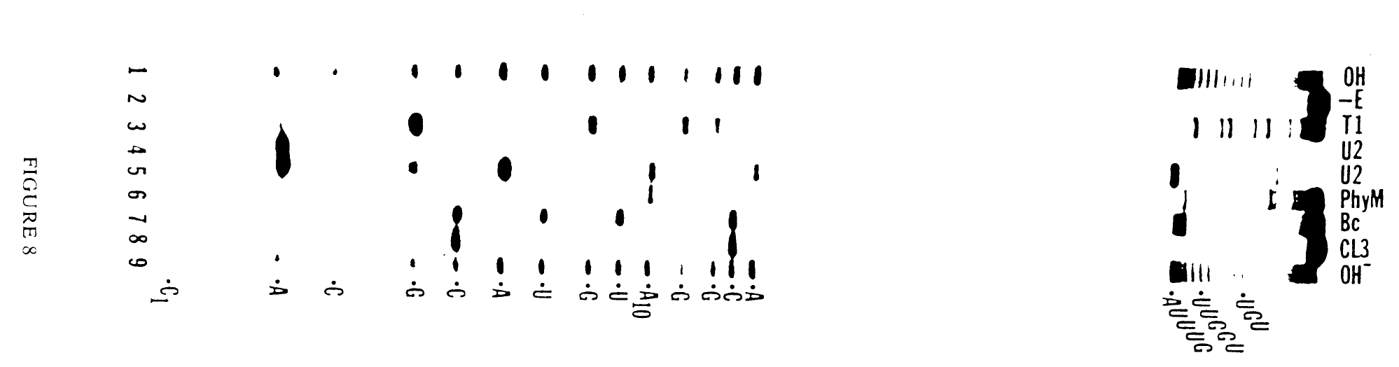

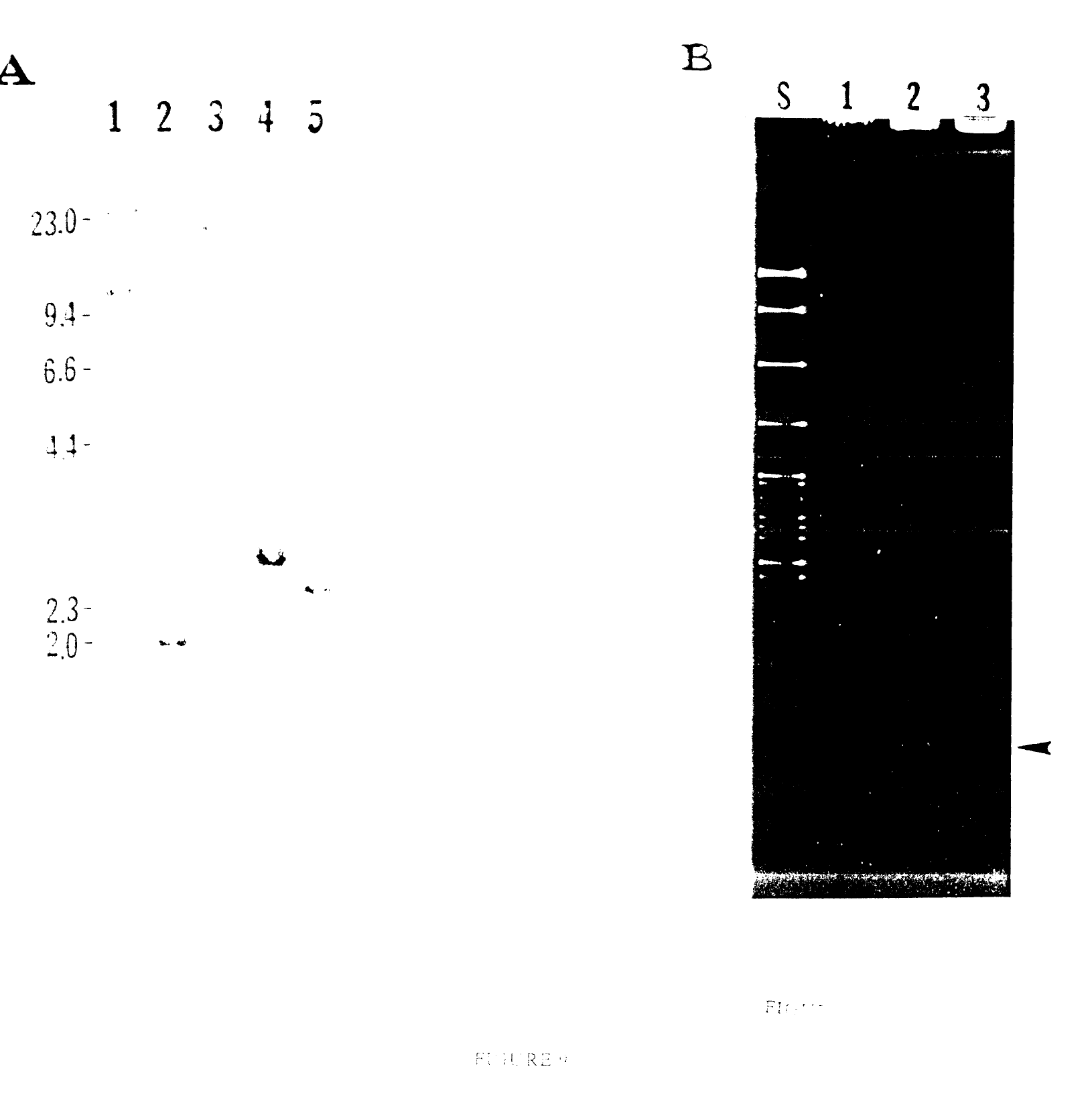

FIG. 8. Determination of the RNA nucleotide sequence for the branched RNA linked to msDNA. Total RNA was prepared from the clinical strain Cl-1 and fractionated on a 5% acrylamide gel. msDNA containing full length RNA was eluted from the gel. This fraction was then labeled at the 5′ end of the RNA with |γ ³²P|ATP and T4 polynucleotide kinase. The 5′ end labeled RNA linked to msDNA was again purified on an 18% acrylamide-8M urea sequencing gel. The labeled RNA was then sequenced using limited digestion with base-specific RNases as described previously (Dhundale et al., 1987). Lanes: OH partial alkaline hydrolysis ladder; (0.5 M sodium bicarbonate/carbonate pH9.2);-E. no enzyme treatment of the labeled RNA linked to msDNA: T1, RNase T1 (1U/reaction, 55°, 15 min.): U2, RNase U2 (1U and 0.5U/reaction, 55°, 15 min.): PhyM, RNase PhyM (1U/reaction, 55°, 15 min): Bc, RNase B. cerus (2U/reaction, 55°, 15 min.): CL3, RNase CL3 (2U/reaction, 37°, 15 min.). The large gap in the sequence gel is due to msDNA linked at the rG residue position 15 by a 2′, 5′ phosphodiester linkage (Furuichi et al., 1987). The RNA sequence at the 3′-end region from the branched rG residue (the upper part of the gel) was determined from 6% gel (data not shown).

FIG. 9. Southern blot analysis of E. coli Cl-1 chromosomal DNA(A) and analysis of msDNA synthesis by pLI-1E and pCl-1P(B). A: The chromosomal DNA was digested with EcoRI (lane 1), HindIII (lane 2). BamHI (lane 3), PstI (lane 4), and BgIII (lane 5). For each lane, 3 μg of the DNA digest was applied to a 0.7% agarose gel. After electrophoresis the gel was blotted to a nitrocellulose filter, and hybridization analysis was carried out according to Souther (Southern, 1975) using msDNA labeled by AMV-RT with |α-³²P|dCTP as a probe. Numbers at the left represent the molecular weights in kb. B: Total DNA prepared from each strain was treated with RNase A. separated on a 5% acrylamide gel and stained with ethidium bromide. Lane S, pBR322 digested with MspI used for molecular size markers; lane 1, DNA prepared from the host strain CL-83(recA⁻); lane 2. CL-83 (recA⁻) transformed with plasmid pCl-1E (11.6 kb Eco RI fragment; see FIG. 5); lane 3. with plasmid pCl-1P (12.8-kb PstI(a)-PstI(b) fragment; see FIG. 5). An arrow indicates the position of msDNA.

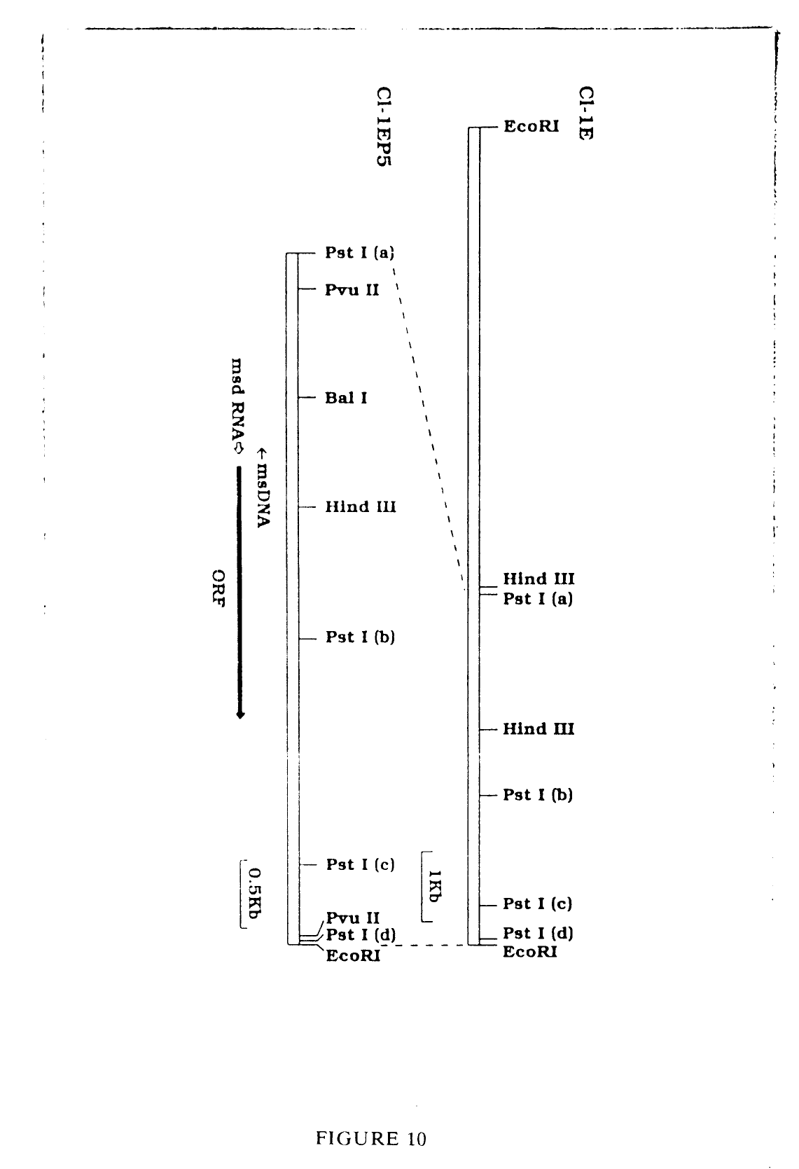

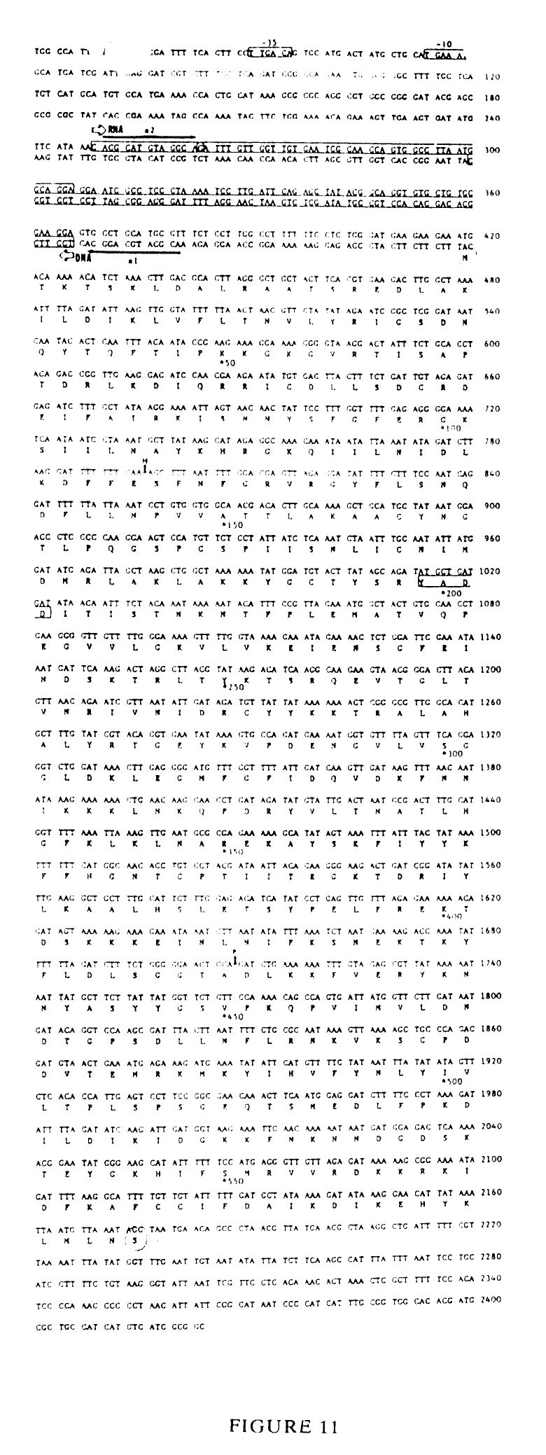

FIG. 10. Restriction map of the 11.6-kb EcoRI fragment. In the Cl-1E map, the left-hand half (EcoRI to HindIII) was not mapped. In the Cl-1EP5 map, the locations and the orientations of msDNA and msdRNA are indicated by a small arrow and an open arrow, respectively. A large solid arrow represents an ORF and its orientation.

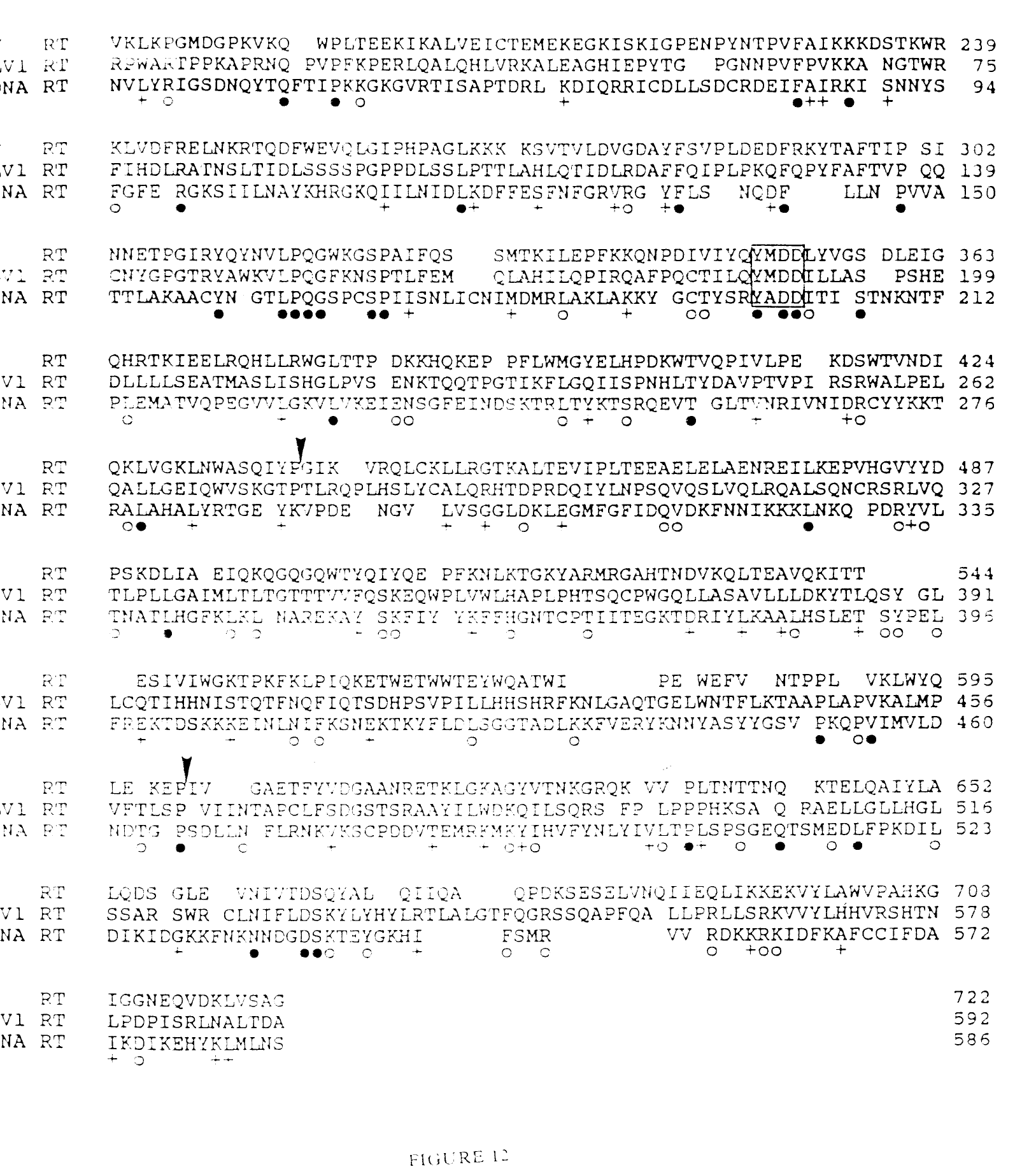

FIG. 11. Nucleotide sequence of the region from the E. coli Cl-1 chromosome encompassing the msDNA and msdRNA coding regions and an ORF downstream of the msdRNA region. The entire upper strand beginning at the Ball site (see FIG. 5) and ending just beyond the ORF is shown. Only a part of the complementary lower strand is shown from base 241 to 420. The long boxed region of the upper strand (249-306) corresponds to the sequence of the branched RNA (msdRNA, see FIG. 7) portion of the msDNA molecule. The boxed region of the lower strand corresponds to the sequence of the DNA portion of msDNA (see FIG. 7). The starting site for DNA and RNA and the 5′ to 3′ orientations are indicated by large open arrows. The msdRNA and msDNA regions overlap at their 3′ ends by 7 bases. The circled G residue at position 263 represents the branched rG of RNA linked to the 5′ end of the DNA strand in msDNA. Long solid arrows labeled a1 and a2 represent inverted repeat sequences proposed to be important in the secondary structure of the primary RNA transcript involved in the synthesis of msDNA (Dhundale et al., 1987). Note that the nucleotide at position 275 (U on the RNA transcript) and the nucleotide at position 373 (G on the RNA transcript) form a U-G pair in the stem between sequence a1 and a2. The proposed promoter elements (−10 and −35 regions) for the primary RNA transcript are also boxed. The ORF begins with the initiation codon at base 418. Single letter designations are given for amino acids. The YXDD amino acid sequence conserved among known RT proteins is boxed. Numbers on the right hand column enumerate the nucleotide bases and numbers with a* enumerate amino acids. Small vertical arrows labeled H and P locate the HindIII and PstI restriction cleavage sites, respectively. The DNA sequence was determined by the chain termination method (Sanger et al., 1977) using synthetic oligonucleotides as primers.

FIG. 12. Amino acid sequence alignment of the E. coli msDNA ORF with a portion of the retroviral Pol sequence from HIV and HTLV1. Amino acid sequences are compared with matching residues assigned as follows: (←) amino acid common to msDNA and HIV RTs; (o) amino acid shared by msDNA and HTLV1 RTs; and (o) amino acid shared by all three proteins. Arrows divide the protein sequences into three functional domains (Toh et al., 1983; Geng et al., 1985; Varmus, 1985, Tanese et al., 1988): An amino terminal RT domain, a carboxy terminal RNase H region, and a central “tether” region. The specific amino acid residues for the RT, tether, and RNase H domains, for each protein are: HIV, 177-439, 440-600, 601-722 respectively; HTLV1, 15-277, 278-462, 463-592 respectively; msDNA ORF, 32-290, 291-465, 466-586 respectively. The YXDD polymerase consensus sequence is outlined with a box.

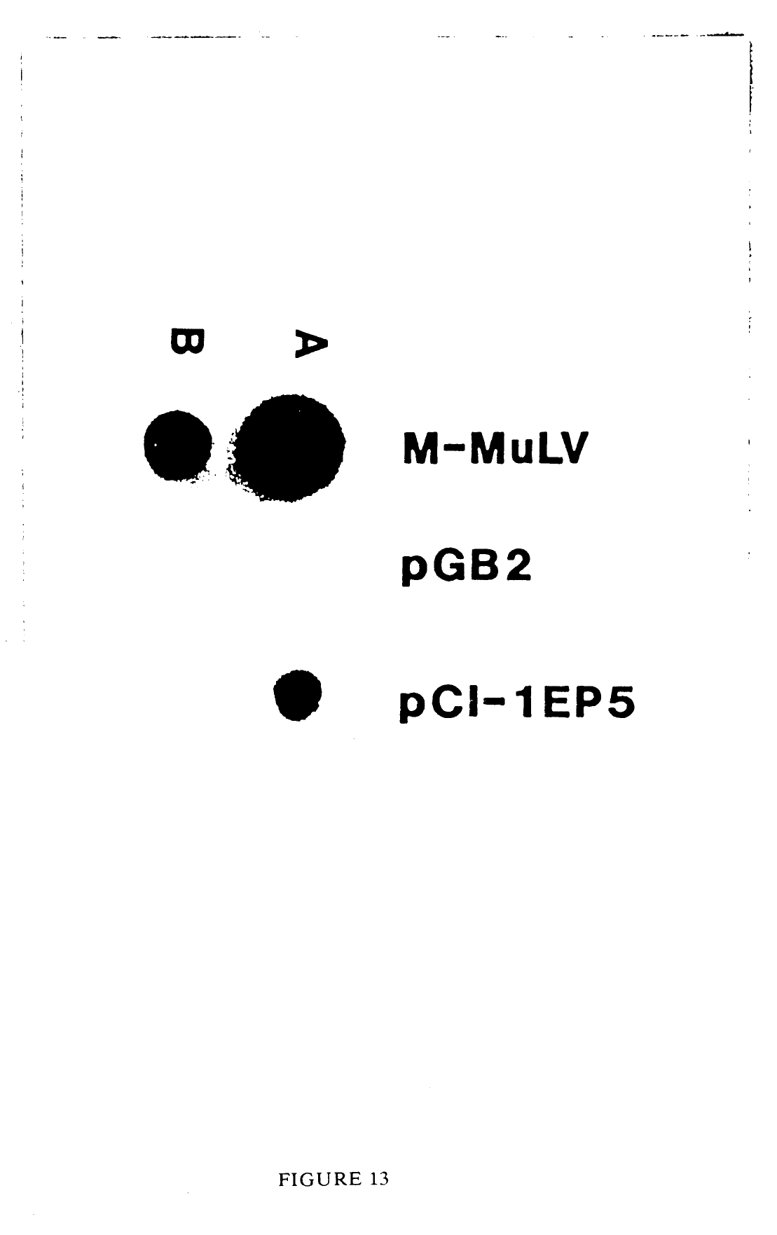

FIG. 13. Detection of RT activity from various cell extracts. Crude cell extracts were prepared from E. coli strain C2110 (polA) (Tanese et al., 1985; Tanese et al., 1986. E. coli strain C2110 (polA1⁻) was a gift from M. Roth and S. Goff) containing plasmid pCl-1EP5 encoding the msDNA-ORF (see FIG. 10) as well as the vector plasmid (pUC9; Yanisch-Perron et al., 1985) alone. Extracts were also prepared from the E. coli strain PRTS7-1 (polA+) containing the cloned M-MuLV RT gene (Varmus et al., 1985; Tanese et al., 1977; Tanese et al., 1985; Tanese et al., 1986. Crude extracts were prepared essentially as described (Roth et al., 1985; Hizi et al., 1988). Crude extract equivalent to 15 μg total protein was added to a 50 μl reaction cocktail (50 mM tris-HCl pH7.8, 10 mM DTT, 60 mM NaCl, 0.05% NP-40, 10 mM MgClhd 2. 0.5 μg poly(rC)-oligo(dG), and 0.1 μM |α-³²P|dGTP and incubated at 37° C. for one hour. Five μl of the reaction mixture was then spotted onto DEAE paper (DE81; Whatman Inc.). The paper was washed to remove unincorporated label (Tanese et al., 1985; Tanese et al., 1986) and then exposed to an X-ray film. In row (A) all reactions contain added template primer (poly rG-dG). Row (B) contains control reactions in which no template-primers added. Columns contain the designated cell extracts: M-MuLV, cloned Moloney Murine Leukemia Virus RT gene: pGB2 (Churchward et al., 1984), vector plasmid in strain C2110;pCl-1EP5, recombinant plasmid with the cloned msDNA gene. The large amount of background activity observed with the M-MuLV control extract is due to the activity of DNA Polymerase I since this extract is obtained from a PolA⁺ strain (HB101).

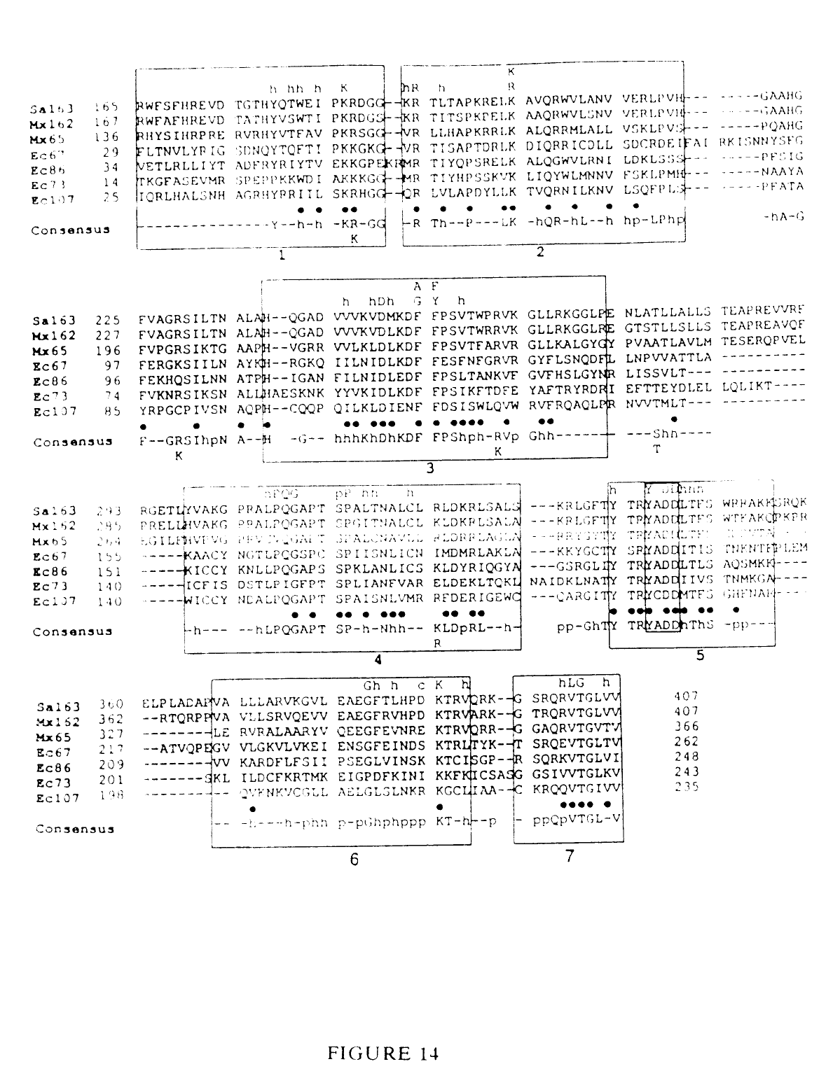

FIG. 14 shows the amino acid sequence alignment of bacterial RT carried out according to Xiong and Eickbush (1990). Amino acids highly conserved in eukaryotic RTs are shown at the top of the sequences. These amino acids include largely unvaried residues or chemically similar residues. (h) Hydrophobic residue; (p) small polar residues; (c) charged residue. Amino acids conserved in all seven bacterial RTs (identical residues plus functional conserved residues indicated by h for hydrophobic residues or p for polar residues) are marked by solid dots at the bottom of the sequences. The consensus sequence shown at the bottom of the sequences is determined when five out of seven sequences contain an identical or a chemically similar residue (h, hydrophobic residue; p, charged and polar residue). The subdomains 1 to 7 are according to Xiong and Eickbush (1990), which are boxed and indicated by numbers. The highly conserved YXDD sequences are also boxed. Numbers on the right indicate the amino acid positions from the amino terminus for each RT. Sources for the sequences are Sal63 (Hsu et al., 1992b), Mx162 (Inouye et al. 1989), Mx65 (Inouye et al. 1990), Ec67 (Lampson et al. 1989b), Ec86 (Lim and Maas 1989), Ec73 (Sun et al. 1991), and Ec107 (Herzer et al. 1992).

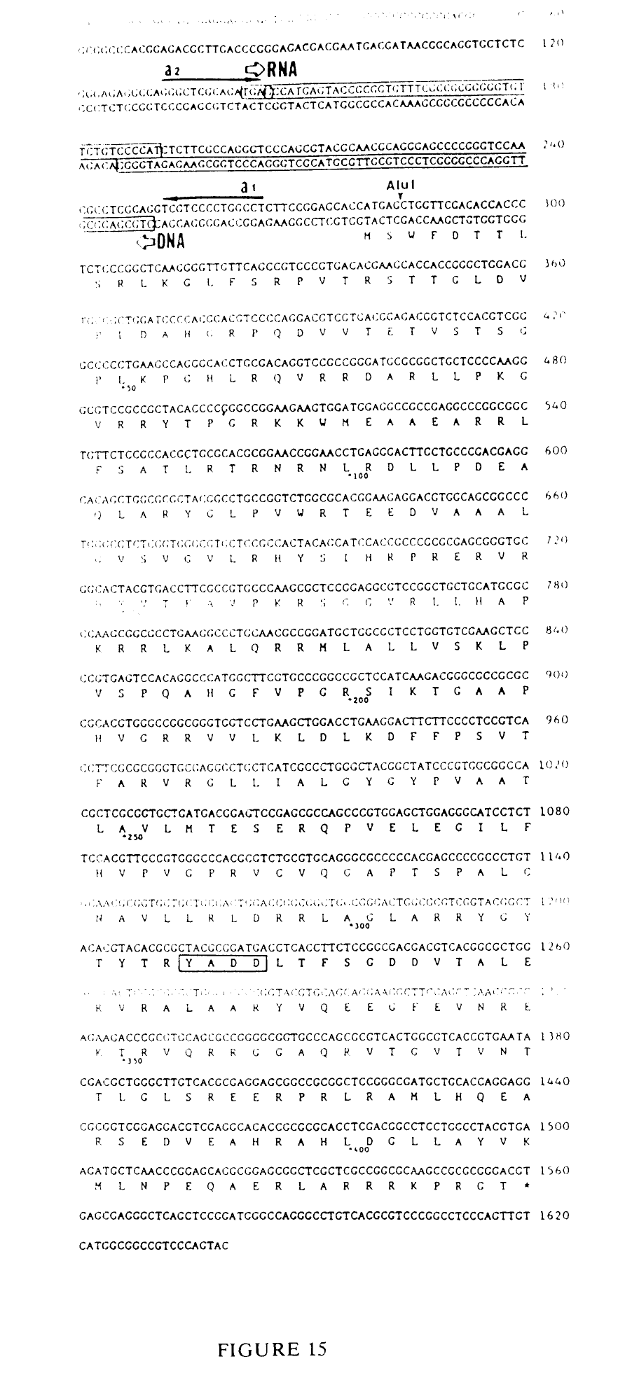

FIG. 15 shows nucleotide sequence of the chromosomal region encompassing the Mx65-msDNA and msdRNA coding regions and an ORF region downstream of msr. The sequence covers from the AluI(A) site to 78 bp downstream of the ORF. The complementary strand is only shown from bases 121-300. The boxed region of the upper strand (positions 143-191) and the boxed region of the lower strand (positions 186-250) correspond to the sequences of msdRNA and msDNA, respectively. The starting sites for DNA and RNA and the 5′ to 3′ orientation are indicated by open arrows. The msdRNA and msDNA regions overlap at their 3′ ends by 6 bases. The circled G residue at position 206 represents the branched guanosine of RNA linked to the 5′ end of the DNA strand in msDNA. Long solid arrows labeled a1 and a2 represent inverted repeat sequences proposed to be important in the secondary structure of the primary RNA transcript involved in the synthesis of msDNA. The ORF begins with the initiation codon at base 279. The YXDD amino acid sequence highly conserved among known RT proteins is boxed. Numbers on the right-hand column enumerate the nucleotide bases, and numbers with asterisks enumerate amino acids (single-letter code). The DNA sequence was determined by the chain-termination method using synthetic oligonucleotides as primers.

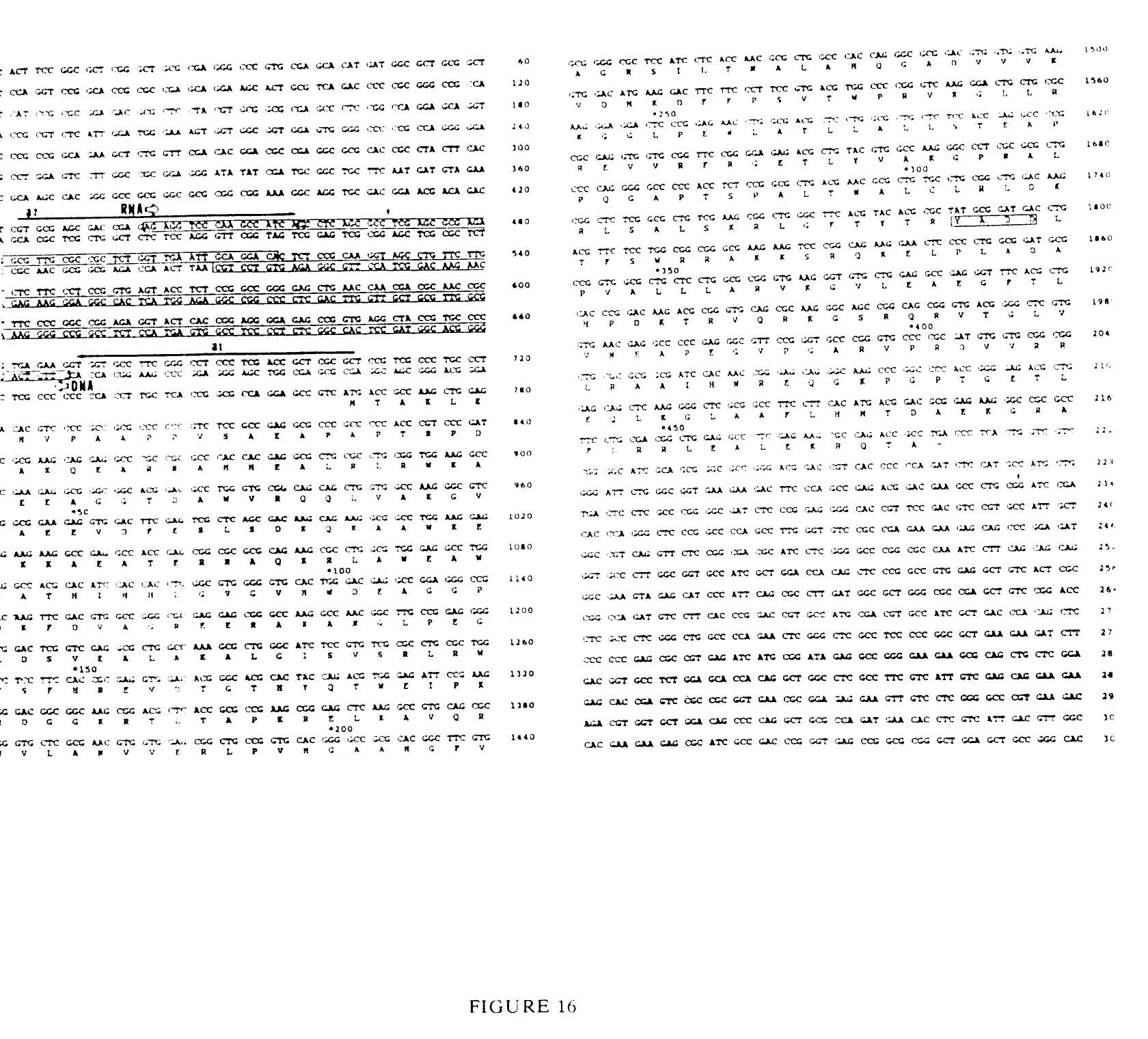

FIG. 16 shows nucleotide sequences of 3,060 bases encompassing msr, msd, and the RT gene of S. aurantiaca. The sequence from base 421 to base 720 which contains msr and msd is shown double stranded. The boxed regions of the upper strand (bases 440 to 540) and the lower strand (bases 508 to 670) correspond to the sequences of msdRNA and msDNA, respectively. The starting sites for msDNA and msdRNA are indicated by open arrows. The circled G at the position 458 is the branched rG of msdRNA linked to the 5′ end of msDNA. Long solid arrows labeled with a1 and a2 represent inverted repeated sequences proposed to form the secondary structure in the primary RNA transcript which serves to prime msDNA synthesis. Amino acids are indicated by single letters. The YXDD sequence highly conserved among known RTs is boxed. X^{e} and B^{f} sites are indicated by arrows. Numbers on the right-hand side and numbers with asterisks represent numbers for bases and amino acids, respectively.

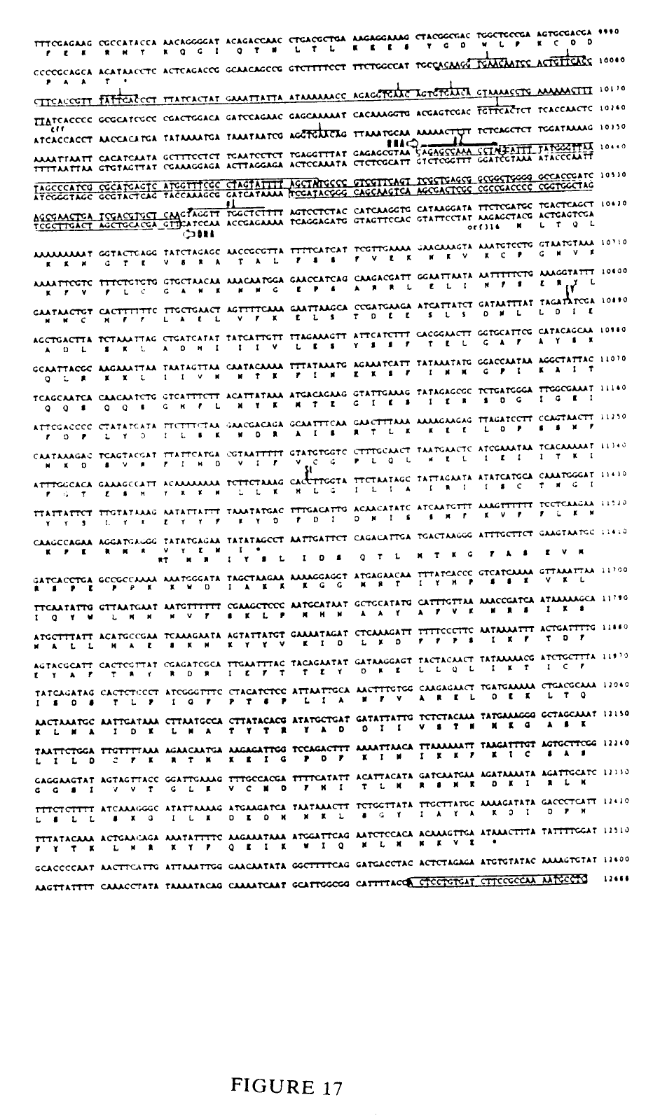

FIG. 17 shows the sequences of msdRNA and msDNA which are boxed and their orientations are indicated by open arrows. The branched G residue at position 10425 is circled. The inverted repeat sequences require for the biosynthesis of msDNA-Ec73 are shown by arrows labeled a1 and a2. Amino acid residues of Ec73-RT are shown by a single-letter code put at the center of each codon.

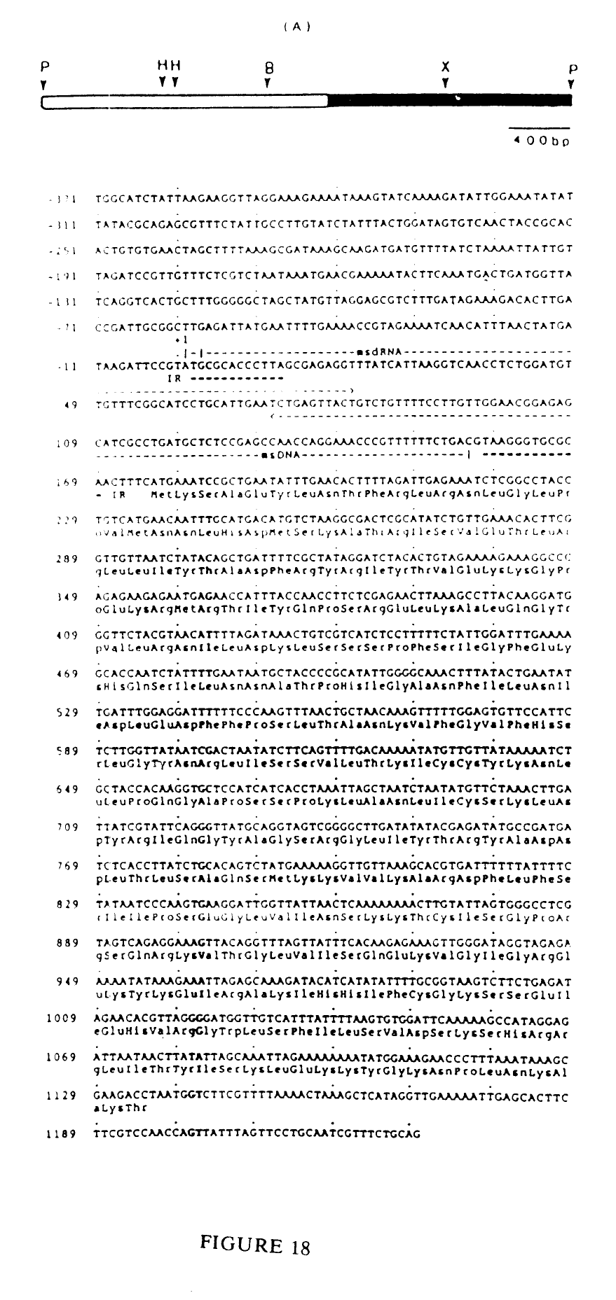

FIG. 18 shows the restriction map of the 3.5 kb insert of pDB808 and nucleotide sequence of chromosomal determinants of the msDNA-RNA compound of E. coli B. (A) Restriction map of the 3.5 kb insert of clone pDB808. The solid bar represents the region whose sequence is presented in (B). Transcription is from left to right. Restriction enzymes are: P, PstI, H, HpaI; B, BgIII; X, XhoI. (B) Nucleotide sequences of the chromosomal determinants. Only the strand corresponding to the transcript is shown. Nucleotides are numbered starting from the first base observed in the msdRNA. The mdsRNA coding region is overlined, and the msDNA coding region is underlined. The msDNA sequence is complementary to the sequence shown in this figure. Inverted repeats are indicated by double-dashed lines. The G at position 14 is the branched guanylate of msdRNA in the msDNA RNA compound. IR, 12 bp inverted repeat.

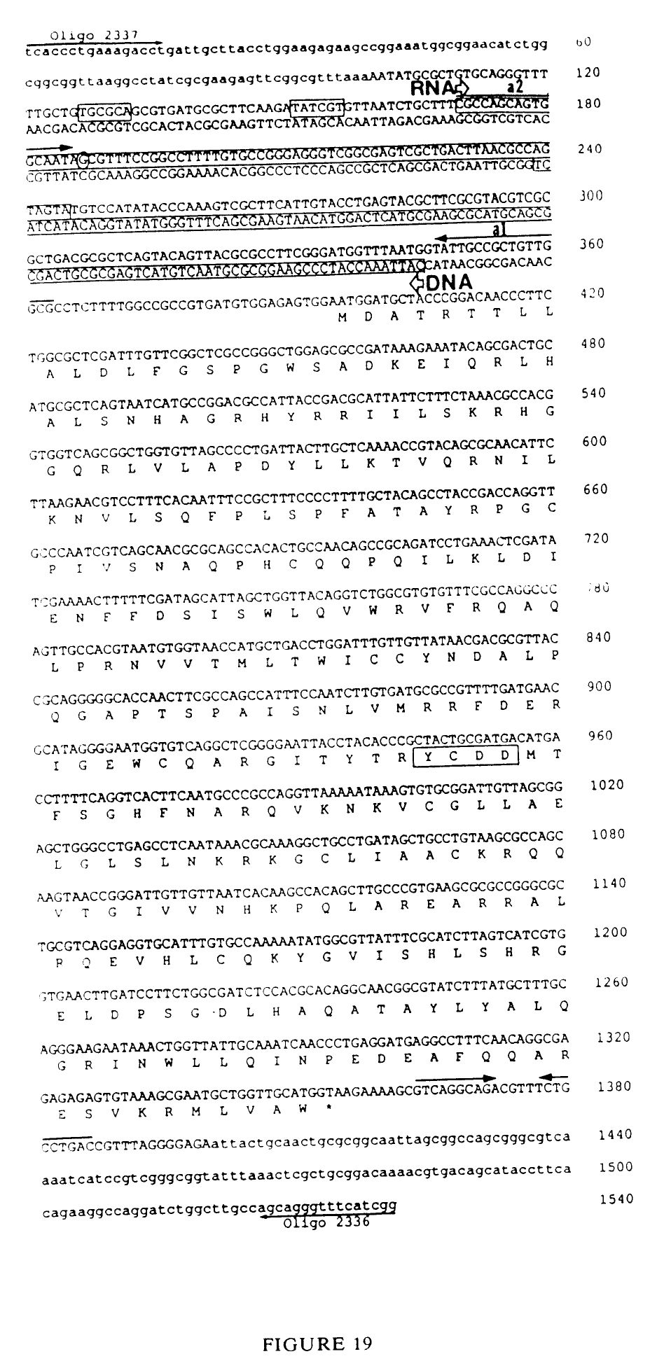

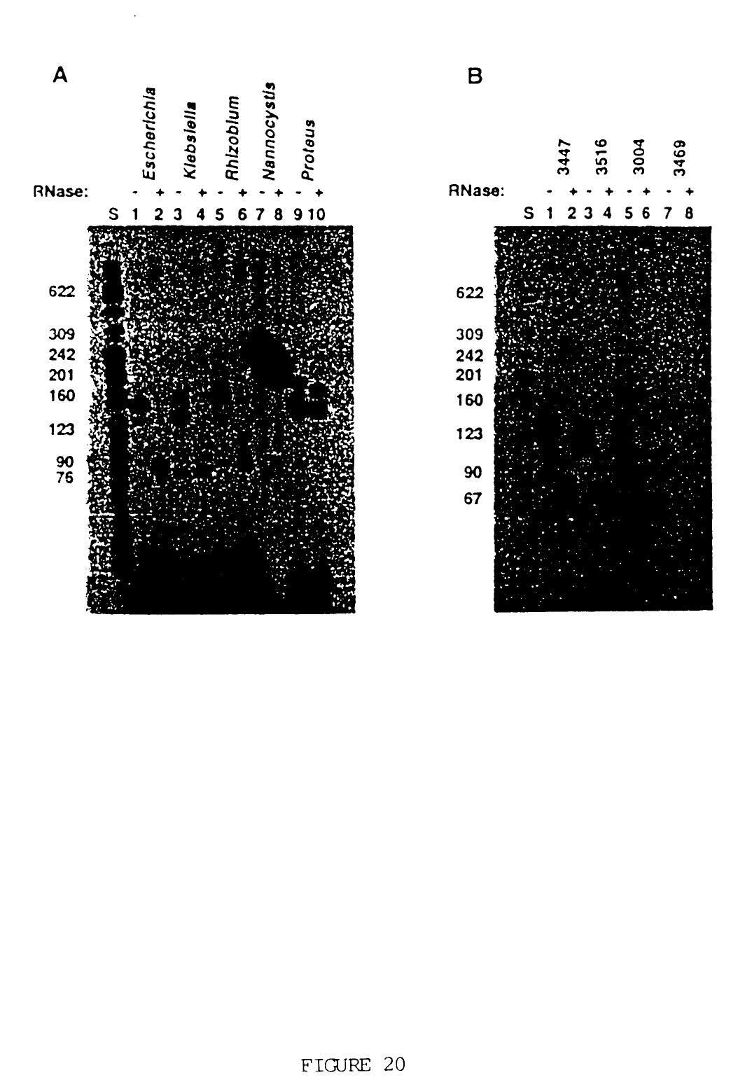

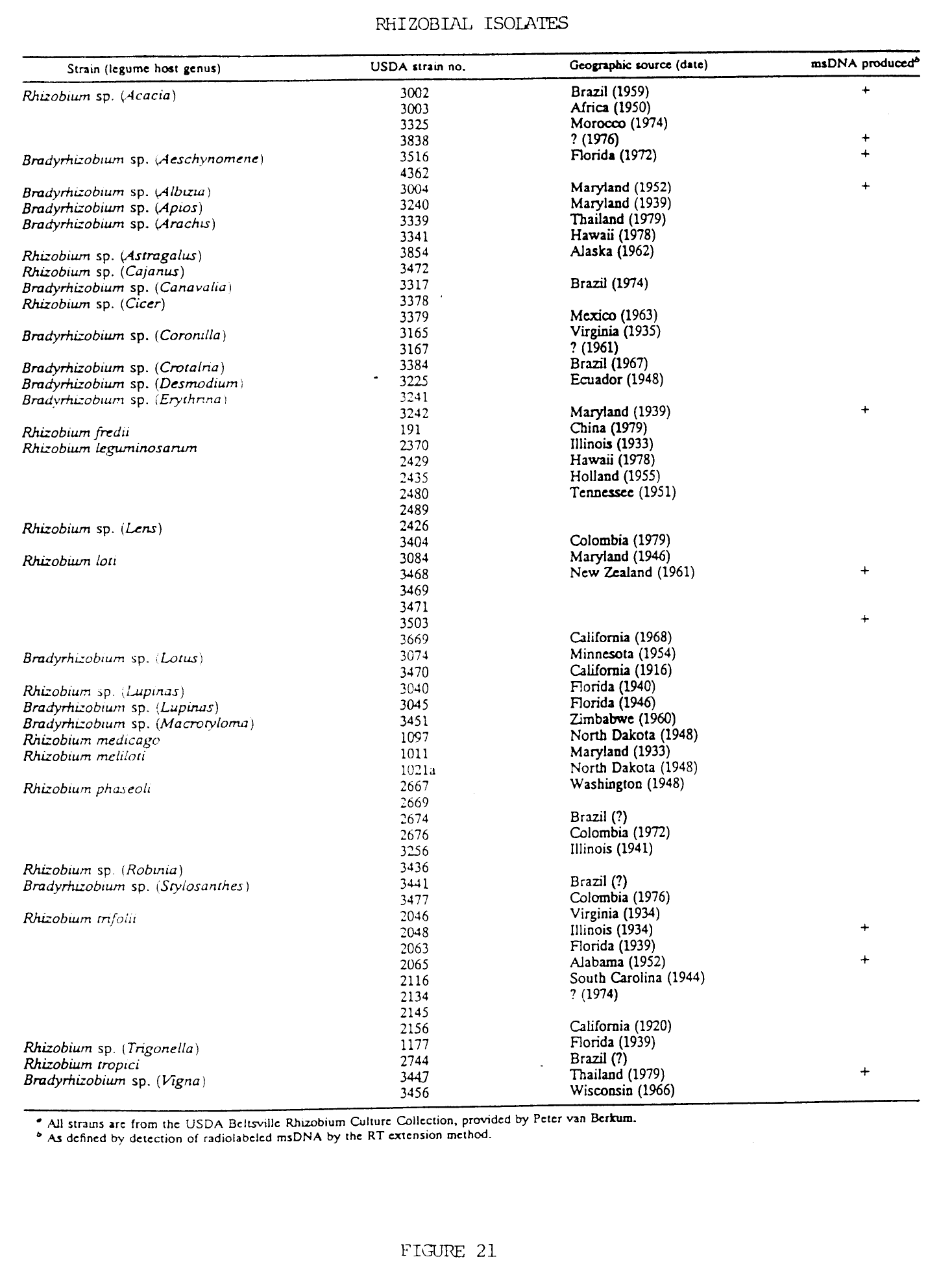

FIG. 19 shows sequences of the retron and flanking regions of Ec107. The sequences corresponding to the K-12 genomic DNA are shown in lower case letters from bases 1-99 and 1400-1540. The msRNA and msDNA regions are boxed. Also indicated are the a1-a2 conserved inverted repeats (indicated by arrows) and the branched G, which is circled. The RT consists of 319 amino acids and contains the YXDD sequence (boxed) which is highly conserved among known RTs. The transcription start site occurs at base 170; a possible terminator is indicated by head-to-head arrows following the RT coding region. Primer extension was utilized in order to determine the transcription start site. These sequence data will appear in the EMBI/GenBank/DDJB Nucleotide Sequence Data Libraries under the accession number X62583.

### DETAILED DESCRIPTION OF THE INVENTION

The description which follows describes msDNA and RT from Myxococcus xanthus. This is a typical bacterium which belongs to a genus of bacteria, whose representative members possess an RT capable of synthesizing msDNA.

The existence of a peculiar branched RNA-linked DNA molecule called msDNA (multicopy single-stranded) has been demonstrated in various myxobacteria, Gram-negative soil bacteria (Yee et al., 1984; Dhundale et al., 1985; Furuichi et al., 1987a,b; Dhundale et al., 1987; Dhundale et al., 1988b). msDNA-Mx162) from Myxococcus xanthus consists of 162-base single stranded DNA, the 5′ end of which is linked to the 2′ position of the 20th rG residue of a 77-base RNA molecule (msdRNA) by a 2′, 5′-phosphodiester linkage (Dhundale et al., 1987). It exists at a level of approximately 700 copies per genome. Stigmatella aurantiaca also possesses an msDNA (msDNA-Sal63) which is highly homologous to msDNA-Mx162 (Furuichi et al., 1987b). In addition to msDNA-Mx162, M. xanthus has another smaller species of msDNA (mrDNA or msDNA-Mx65), which has no primary sequence homology with msDNA-Mx162 or msDNA-Sal63 (Dhundale et al., 1988b). However, all msDNAs so far characterized share key structural features such as a branched rG residue, stem-and-loop structures in RNA and DNA molecules, and a DNA-RNA hybrid at the 3′ ends of DNA and RNA molecules.

Previously it was predicted that reverse transcriptase is required for msDNA biosynthesis on the basis of the finding that msdRNA is derived from a much longer precursor, which can form a very stable stem-and-loop structure (Dhundale et al., 1987). This precursor molecule was proposed to serve as a primer for initiating msDNA synthesis as well as a template to form the branched RNA-linked msDNA. The latter reaction requires reverse transcriptase activity. In M. xanthus, the region coding for the RNA molecule (msr) is located on the chromosome in the opposite orientation to the msDNA coding region (msd) with the 3′ ends overlapping by 6 bases for msDNA-Mx65 (Dhundale et al., 1988b) or by 8 bases for msDNA-Mx162 (Dhundale et al., 1987). In addition, as in all the msDNAs found in myxobacteria, there is an inverted repeat comprised of a 14-base sequence for msDNA-Mx65 (Dhundale et al., 1988b) or a 34-base sequence for msDNA-Mx162 (Dhundale et al., 1987) and a 33-base sequence for msDNA-Sal63 (Furuichi et al., 1987b) immediately upstream of the branched G residue and a sequence immediately upstream of the msDNA coding region. As a result of this inverted repeat, a longer primary transcript beginning upstream of the RNA coding region and extending through the msDNA coding region is considered to self-anneal and form a stable secondary structure. When three base mismatches were introduced into the secondary structure immediately upstream of the branched rG residue, msDNA synthesis was almost completely blocked. However, if three additional base substitutions were made on the other strand to resume the complementary base pairing, msDNA production was restored (Hsu et al., 1989). This result strongly supports the proposed model for msDNA synthesis.

It was also shown that a deletion mutation at the region 100 base pairs (bp) upstream of the DNA coding region (msd) and an insertion mutation at a site 500 bp upstream of msd caused a significant reduction in msDNA production (Dhundale et al., 1988a). This indicates that there is a cis- or trans-acting positive element required for msDNA synthesis in this region. In this report we determined the DNA sequence of this region and found an opening reading frame (ORF) coding for 485 amino acid residues beginning with an initiation codon, ATG, which is located 77 bp upstream of msd (or 231 bp downstream of msr). The very close proximity between msd and the ORF suggests that they may be transcribed as a single transcript. The amino acid sequence of the ORF shows similarity with retroviral reverse transcriptases. We discuss a possible origin of the reverse transcriptase gene as well as a possible relationship between the msDNA system and retroviruses. Recently, some strains of Escherichia coli were found to produce msDNA and the gene for reverse transcriptase which is essential for msDNA production, is linked to the msd region, (Lim and Maas, 1989: Lampson et al., 1989b). Comparison of the msDNA systems of M. xanthus and E. coli raises an intriguing question as to how the extensive diversity found in msDNA systems has emerged in bacteria and what possible functions msDNA may have.

In a preceding paper, it was demonstrated that msDNA is in fact synthesized by reverse transcriptase in a cell-free system in M. xanthus (Lampson et al., 1989a).

Reverse transcriptases are isolated, and if desired, purified, and biological characterization carried out, if desired, by known methods such as those described in Lampson, B. C., M. Viswanathan, M. Inouye and S. Inouye, “Reverse Transcriptase from Escherichia coli Exists as a Complex with msDNA and is Able to Synthesize Double-stranded DNA”, J. Biol. Chem. 265:8490-8496 (1990), which is incorporated by reference as if fully set forth herein.

### RESULTS AND DISCUSSION

### Identification of an ORF Associated with msd

On the basis of mutations closely associated with msd which significantly reduce msDNA production, it was assumed that in this region there is a cis- or trans-acting element which is essential for msDNA synthesis (Dhundale et al., 1988a). FIG. 1 shows a restriction map around msd. The msDNA coding region is shown by a thin arrow from right to left (msd), and the msdRNA coding region by a thick open arrow (msr). In the previous work (Dhundale et al., 1988a), two mutations were constructed; one, a deletion mutation in which the sequence from AluI(b) to SmaI was replaced by a gene for kanamycin resistance (see FIG. 1), and the other an insertion mutation at the SmaI site by a gene for kanamycin resistance (see FIG. 1).

In order to elucidate the properties of the element required for msDNA production, the DNA sequence of the region upstream of msd was determined as shown in FIG. 2. A long open reading frame (ORF) beginning with an initiation codon was found 77 bases upstream of msd. The ORF is preceded by a ribosome binding sequence of AGG (residue 630 to 632) 7 upstream of the initiation codon. The ORF codes for a polypeptide of 485 amino acid residues. The AluI(b) and SmaI sites (see FIG. 1), where mutations inhibiting msDNA synthesis were created, are located at amino acid residue-12 and-142 of the ORF, respectively or at the nucleotide sequence from residue-672 to-675, and from residue-1061 to-1066, respectively (FIG. 2). In FIG. 2, msd or the DNA sequence corresponding to the msDNA sequence is indicated by the closed box on the lower strand and the orientation is from right to left. Similarly, the msdRNA sequence (msr) is also indicated by the closed box on the upper strand and the orientation is from left to right. The msd and msr regions overlap by 8 bases. An inverted repeat is also indicated by arrows with letter a1 and a2. This inverted repeat comprises a 34-base sequence immediately upstream of the branched G residue (residue 317 to 350; sequence a2 in FIG. 2) and another 34-base sequence at the 3′ end (residue 597 to 564; sequence a1). This inverted repeat is essential to form a stem structure which provides a stable secondary structure in a long primary transcript. This secondary structure is considered to serve as the primer as well as the template for msDNA synthesis (Dhundale et al., 1987; Hsu et al., 1989).

### Sequence Similarity with Retroviral Reverse Transcriptases

When the amino acid sequence of the ORF was compared with known proteins, a striking similarity was found between the sequence from Leu-308 to Ser351 and retroviral reverse transcriptases (RT). In particular, this region contains the YXDD sequence, the highly conserved sequence in all known RTs. This sequence (Tyr-344 to Asp-347) is boxed in FIG. 2. In FIG. 3, the ORF sequence of 266 amino acid residues from Ala-170 to Lys-435 is compared with RTs from HIV (human immunodeficiency virus: Ratner et al., 1986) and HTLV1 (human T-cell leukemia virus type 1; Seiki et al., 1983). As mentioned above, within the sequence of 44 amino residues from Leu-308 to Ser-351, there are 14 and 12 identical residues with HIV (32%) and HTLV1 (27%), respectively. The entire RT domains of HIV and HTLV can also be aligned with the ORF sequence from Ala-170 to Lys-435, with much less similarity as shown in FIG. 3. However, the same region was found to be extremely well aligned with the RT which was recently found in a clinical strain of Escherichia coli (Lampson et al., 1989b). This E. coli RT consists of 586 amino acid residues, and its amino terminal domain (residue-32 to-291) and the carboxyl terminal domain (residue-466 and-586) have been demonstrated to have sequence similarity with retroviral RT and ribonuclease H. This RT gene from E. coli was shown to be required for the production of msDNA (msDNA-Ec67) and to have reverse transcriptase activity (Lampson et al., 1989b). FIG. 3 shows that the sequence similarity between E. coli and M. xanthus RTs is distributed within almost the entire RT region; in particular in the region from Tyr-181 to Ser-212. 15 out of 32 residues are identical (47% similarity); in the region from Gly-226 to Gly-265, 19 out of 40 residues (48% similarity); in the region from Leu-308 to 351, 26 out of 44 residues (59% similarity); and in the region from Lys-354 to Asn-408, 21 out of 55 residues (38% similarity). Overall, similarity from Ala-170 to Lys-435 is 32% (85 out of 266 residues are identical). In spite of these similarities, the M. xanthus ORF does not have the domain, which shows apparent sequence similarity with ribonuclease H (RNase H). The RNase H domain is found to be located in the carboxyl terminal region of the same polypeptide in which the RT domain exists in the amino terminal region in the case of the E. coli RT and other retroviral RTs. In the preceding paper, it was shown that there is a precise coupling between RT and RNase H activity (Lampson et al., 1989a). Therefore RNase H may still reside with the ORF, or RNase H may be encoded by a separate gene.

### Sequence Similarity with Other Proteins

In contrast to the E. coli RT and other retroviral RTs, the ORF found in M. xanthus has a long amino terminal extra domain consisting of approximately 170 residues. Interestingly, this region shows some sequence similarities with the carboxyl terminal region associated with integration protein of Mo-MLV (Moloney murine leukemia virus; Shinnick et al., 1981) (see FIG. 4A); the sequence from Pro-18 to Leu-128 of the ORF shows 22% similarity (24 out of 111 residues) with the region from Pro-1070 to Leu-1179 of the gag-pol polyprotein of Mo-MLV. It should be noted that this region of Mo-MLV is unique for Mo-MLV integration protein and does not share sequence similarity with other retroviral endonucleases (Johnson et al, 1986). It is also interesting to notice that in Ty retrotransposon, this domain is located in front of the RT domain in contrast to the retroviral endonuclease domain (Clare and Farabaugh, 1985).

As pointed out above, the ORF does not have homology to E. coli or retroviral RNase H. Instead, it has a short sequence of approximately 80 residues after the RT domain. In this region, one can also find sequence similarity with a part of the gag region of HIV. As shown in FIG. 4B, the sequence from Gly-411 to Glu-485 has 22 identical amino acid residues (31% similarity) with the region from Gly-396 to Pro-461 of the gag protein of HIV (Ratner et al, 1985).

### Requirement of Reverse Transcriptase

The fact that disruption of the ORF significantly reduced msDNA production in M. xanthus (Dhundale et al., 1988a) and the fact that the ORF has sequence similarity with retroviral RTs strongly supports the previous hypothesis that RT is required for the synthesis of msDNA (Dhundale et al., 1987). Recently, we were able to demonstrate that msDNA is indeed synthesized by reverse transcriptase activity in a cell-free system (Lampson et al., 1989a). The fact that a small amount of msDNA (3% of the wild type level) is still produced in the ORF mutants (Dhundale et al., 1988a) is most likely due to another RT associated with smaller msDNA (msDNA-Mx65; previously assigned mrDNA: Dhundale et al., 1988b). In fact, an ORF has been found to be associated with the region responsible for msDNA-Mx65 production.

At present it is unknown if the ORF is transcribed together with msdRNA from a common upstream promoter or if the ORF has its own independent promoter. Previously, a major RNA transcript of approximately 375 bases by S1 mapping (Dhundale et al., 1987) was identified. This transcript covers the region from approximately 75 bases upstream of msr (at around residue-256 in FIG. 2) to approximately 70 bases upstream of msd (at around residue-632 in FIG. 2). This indicates that this RNA transcript ends at the ribosome binding site (AGG, 630-632) of the ORF. It is possible that the primary RNA transcript covers not only the msr-msd region but also the entire ORF. This transcript of approximately at least 2 kilobase (kb) is then used as the mRNA for the ORF to produce RT. At the same time, the 5′ untranslated region of 350 bases forms a stable secondary structure which serves as a primer and a template for msDNA synthesis as previously proposed (Dhundale et al., 1987). Because of the secondary structure, the 5′ end region is probably much more stable than the ORF mRNA region. As a result, only the 375-base RNA from the 5′ end of the transcript was detected in the previous work. In E. coli, the RT gene was shown to be transcribed from a single promoter for the msr region (Lampson et al., 1989b).

### Evolution of Reverse Transcriptase

All of the RTs so far identified are from eukaryotic origins, and associated with either retroviruses or retrotransponsons. DNA synthesis for retroviruses and transposition events for retrotransponsons occur via RNA which is used as a template for RTs (see review by Varmus, 1985). From amino acid similarity in various RTs, possible evolutionary relationships among these RTs has been proposed (Yuki et al., 1986).

The present invention demonstrates that RTs are not specific to eukaryotes but exist in prokaryotes as well. An intriguing question arises as to the evolutionary relationship between prokaryotic and eukaryotic RTs and the origin RT. In order to compare the amino acid sequences of these RTs, the sequence of the M. xanthus RT from Gly-304 to Leu-371 was chosen, since this sequence includes the YXDD box, the most conserved region among different RTs. In FIG. 5A this sequence is compared with 13 other representative RTs from bacteria, yeast, plant, mitochondrial plasmid, and animal retroviruses. Within these 14 sequences, the D-D sequence (residues-346 and-347) is completely conserved, and both G-311 and Y-344 are also well conserved except for Ty-RT. Besides these residues. L-308, P-309, O-310, S-315, P-316, L-330, S-351, and L-371 are fairly well conserved among these sequences. On the basis of the numbers of identical amino acid residues, M. xanthus RT has the following similarities with other RTs: 47% (32 amino acid residues) with E. coli Cl-1 RT; 41% (28) with E. coli B RT; 24% (16) with HIV, BLV, and mitochondrial plasmid RTs; 22% (15) with Mo-MLV RT; 21% (14) with RSV, 17.6, gypsy, and Tal-3 RTs; 19% (13) with HTLV1 RT; 15% (10) with Ty912 RT; and 9% (6) with Copia RT. On the basis of the phylogenetic relationships among RTs proposed by Yuki et at. (1986), and the present data, a dendrogram of homology of various RTs may be constructed as shown in FIG. 5B. As proposed earlier (Yuki et al., 1986), modern RTs are composed to two major groups I and II. One group (group II) consists of retrotransponsons found in yeast (Ty912), plant (Tal-3), and Drosophila (Copia). Bacterial RTs seem to belong to the other group (group I) together with other retrotransponsons from Drosophila such as 17.6 and gypsy, mitochondrial plasmid RT, and retroviral RTs. This indicates that both prokaryotic and eukaryotic RT genes were possibly derived from a single ancestral RT gene.

### Origin of the M. xanthus Reverse Transcriptase

In addition to the sequence similarity between the M. xanthus RT and RTs from retroviruses and retrotransponsons, msDNA shares other interesting similarities with retroviruses and retrotransponsons; msDNA (synthesis of single-stranded DNA) starts at a site 77 bases upstream of the RT gene and the orientation of DNA synthesis is opposite to the direction of translation of the RT gene. In the case of retroviruses and retrotransponsons, single-stranded DNA synthesis proceeds at the 5′-end untranslated region of an RNA molecule which serves as the mRNA for RT as well (Weiss et al., 1985). The orientation of DNA synthesis is also opposite to the direction of translation of the RT gene. In the case of msDNA synthesis in RNA transcript itself serving as a template also serves as a primer by self-annealing to form a stable secondary structure (Dhundale et al., 1987), whereas in the case of retroviruses and retrotransponsons tRNAs are recruited from the cell for the priming reaction. At present it is unknown if branched RNA-linked msDNA is the final product of and unknown function or if it is a stable intermediate leading to other products.

Furthermore, it is of great interest whether the M. xanthus RT is associated with a complex such as virus-like particles such as those found for yeast Ty1 element (Eichinger and Boeke, 1988). In a preliminary experiment, msDNA of M. xanthus exists as a complex with proteins in the cell which sediments as a 228 particle. Characterization of this complex may shed light on questions concerning the relationship between msDNA and retrocomponents as well as the functions of msDNA.

At present, there is no information to support the possibility that msDNA may be a transposable element of an element associated with a provirus (or prophages). It is important to point out that the RT gene from M. xanthus appears to be as old as other genomic genes for the following reasons: (a) Nine independent natural isolates of M. xanthus from various sites (including Fiji Island and eight different sites in the United States) contained mutually hybridizable msDNA (Dhundale et al., 1985). Since under the same hybridization condition, msDNA-Mx162 did not hybridize with msDNA-Sa163 [which has extensive homology in both DNA and RNA sequences with msDNA-Mx162; Dhundale et al., (1987)], the nine independent strains M. xanthus are assumed to contain almost identical msDNA. (b) The codon usage of the Mx-162 RT is almost identical to those found

### REFERENCES

Birnhoim H. C., and J. Dolly, Nucl. Acid Res., 7, 1513-1523 (1979).

Boeke J. D., Gorfinkel C. A., Styles, C. A., Fink G. R., Cell, 40, 491 (1985).

Cairns J., Overbaugh J., Miller S., Nature, 335, 142-145 (1988).

Churchward G., Belin D., Nagaime Y., Gene, 31, 165 (1984).

Clare J., Farabaugh P., Proc. Natl. Acad. Sci. USA, 82, 2829-2833 (1985).

Dhundale A., Furuichi S., Inouye S., Inouye M., J. Bacteriol., 164, 914 (1985).

Dhundale A., Lampson B., Furuichi T., Inouye M., Inouye S., Cell, 51, 1105 (1987).

Dhundale A., Furuichi T., Inouye M., Inouye S., J. Bacteriol., 170, 5620-5624 (1988a).

Dickson C., Eisenman R., Fan H., Hunter, E., Teich, N., Molecular Biology of Tumor Viruses, ed. 2, Cold Spring Harbor Laboratory, N.Y. 513, 648 (1982).

Eichinger D. J., Boeke J. D., Cell, 54, 955-966 (1988).

Fasman G., CRC Handbook of Biochem. and Mol. Biol., Nucleic Acids, Vol (2., 102 (1976).

Furuichi T., Dhundale A., Inouye M., Inouye, S., Cell, 48, 47-53(1987a).

Furuichi T., Inouye S., Inouye M., Cell, 48, 55-62 (1987b).

Hsu M. Y., Inouye S., Inouye M., J. Biol. Chem., 264 (1989).

Inouye S., Franceschini T., Inouye, M. Proc. Natl. Acad. Sci., USA, 80, 6829-6833 (1983).

Inouye, S., Hsu, M. Y., Eagle, S. and Inouye, M., Cell 56:709-717 (1989).

Inouye, S, Herzer, P. J. and Inouye, M., Proc. Natl. Acad. Sci, 87:942-945 (1990).

Johnson M. S., McClure M. A., Feng D. F., Gray J., Doolittle R. F., Proc. Natl. Acad. Sci., USA, 83, 7648-7652 (1986).

Kaiser D., Ann. Rev. Genet, 20, 539-566 (1986).

Komano T., Franceschinti T., Inouye S., J. Mol. Biol., 196, 517-524 (1987).

Lampson B. C., Inouye M., Inouye S., Cell, 56, 701-707 (1989a).

Lampson B. C., Inouye S., Inouye M. “msDNA of Bacteria”, Progress in Nucleic Acid Research and Molecular Biology, Vol. 40, pages 1 et seq.

Lampson B. C., Sun J., Hsu M. Y., Vallejo-Ramierez J., Inouye S., Inouye M., Science, 243, 1033-1038 (1989b).

Lampson, B. C., Viswanathan M., Inouye M. and Inouye S., “Reverse Transcriptase from Escherichia coli Exists as a Complex with msDNA and is Able to Synthesize Double-stranded DNA”, J. Biol. Chem. 265:8490-8496 (1990).

Lim, D. and Maas, W. K., Cell, 56:891-904 (1989).

Lim D., Maas W., Mol. Microbiol., 3, 1141-1144 (1989).

Maniatis T., Fritsch E. F., Sambrook J., Molecular Cloning, A Laboratory Manual, Cold Spring Harbor Laboratory, N.Y. (1982).

Maruyama T., Gojobori, T., Aota S., Ikemura T., Nuc. Acid Res. 14, r151-r189 (1986).

Maxam A. M., Gilbert W., Meth. Enzymol., 65, 499 (1980).

Nargang, F. E., Bell, J. B., Stohl L. L., Lambowitz A. M., Cell, 38, 441-453 (1984).

Perron Y. C., Vieria J., Messing J., Gene, 33, 103 (1985).

Ratner L., et al., Nature, 313, 277-283.

Rice N. R., Stephens R. M., Burny A., Gilden, R. V., Virology, 142, 357-377 (1985).

Romeo J. M., Emson B., Zusman D. R., Proc. Natl. Acad. Sci. USA, 83, 6332-6336 (1986).

Roth M. J., Tanese, N., Goff S. P., J. Biol. Chem., 260, 9326 (1985).

Ruskin B., Green M., Science, 229, 4274 (1987).

Saigo K., Kugimiya W., Matsuo Y., Inouye S., Yoshioka K., Yuki S., Nature, 312, 659-661 (1984).

Sanger F., .Nicklen S., Coulson A. R., Proc. Natl. Acad. Sci., USA, 74, 5463-5467.

Seiki M., Hattori S., Hirayama Y., Yoshida M., Proc. Natl. Acad. Sci, USA, 80, 3618-3622 (1983).

Shinnick T. M., Lerner R. A., Sutcliffe J. G. Nature, 293, 543-548 (1981).

Stavenhagen, J. B., Robins D. M., Cell, 55, 247-254 (1988).

Southern E., J. Mol. Biol., 98, 503 (1975).

Tanese N., Roth M., Goff S. P., Proc. Natl. Acad. Sci, USA, 82, 4944 (1985).

Tanese N., Sodroski J., Haseltine W. A., Goff S. P. J. Virol., 59, 743(1986).

Temin H. M., Cell, 21, 599-600 (1980).

Toh H., Hayashida H., Miyata T., Nature, 305, 827 (1983).

Varmus H. E., Nature, 314, 584-585 (1985).

Vieira J., Messing J., Gene, 19, 259-268 (1982).

Visawanathan M., Inouye M., Inouye S., J. Biol. Chem., 264, 13665-13671 (1989).

Voytas D. F., Ausbel F. M., Nature, 336, 242-244 (1988).

Weiss N., Teich H., Varmus H., Coffin J., RNA Tumor Viruses, Vol. 2, Cold Spring Harbor Laboratory (1985).

Yee T., Furuichi T., Inouye S., Inouye M., Cell, 38, 203-209 (1984).

Yuki S., Ishimaru S., Inouye S., Saigo K., Nucl. Acid Res., 14, 3017-3020 (1986).

We claim:

1. An isolated and purified bacterial reverse transcriptase (RT) which is capable of synthesizing msDNA, which RT comprises a conserved sequence of amino acid residues as follows: tyrosine, x which is alanine or cysteine, and two aspartic acid residues.

2. The bacterial RT of claim 1 which comprises a second conserved sequence of amino acid residues as follows: serine, x which is a hydrophobic residue selected from the group consisting of valine, phenylalanine, leucine and isoleucine, x₁ which is a polar residue selected from the group consisting of threonine, asparagine, lysine and serine and x₂ which is a hydrophobic residue selected from the group consisting of tryptophan, phenylalanine and alanine.

3. The bacterial RT of claim 2 which comprises a third conserved sequence of amino acid residues as follows: asparagine, x which is a hydrophobic residue selected from the group consisting of alanin, leucine and phenylalanine and x₁ which is a hydrophobic residue selected from the group consisting of leucine, valine and isoleucine.

4. The bacterial RT of claim 1 which comprises a fourth conserved sequence of amino acid residues as follows: x which is a polar residue selected from the group consisting of arginine, glutamic acid, lysine, valine and glutamine, a second residue which is valine, a third residue which is threonine and a fourth residue which is glycine.

5. The bacterial RT of claim 1 which has the common subdomains 1 through 7 shown in Table 5.

6. The bacterial RT of claim 1 wherein the conserved sequence is located in subdomain 5 shown in Table 5.

7. The bacterial RT of claim 6 which has a total of 61 conserved amino acid residues.

8. An isolated and purified bacterial RT which comprises a sequence of amino acid residues shown in FIG. 14.

9. An isolated and purified bacterial RT from a bacterium which is capable of synthesizing an msDNA as determined by the reverse transcriptase extension in vitro screening test, which indicates the presence or absence of msDNA in the bacterium.

10. The bacterial RT of claim 9 wherein the bacterium is selected from the group of genera consisting of Myxococcus, Escherichia, Proteus, Klebsiella, Flexabacter, Cytophaga, Stigmatella, Salmonella, Nannocystis, Rhizobium and Bradyrhizobium.

11. The bacterial RT of claim 10 wherein the in vitro screening test for determining the presence or absence of msDNA in the bacterium comprises treating a preparation of total RNA extracted from the bacterium with a reverse transcriptase (RT) in the presence of a radiolabeled deoxynucleotide, which RT, when msDNA is present in the total RNA of the bacterium, utilizes the DNA portion of the msDNA as a primer and the RNA portion of the msDNA as a template for radiolabeling the DNA portion of the msDNA, electrophoresing the treated RNA preparation and determining the presence of msDNA in the bacterium by detecting a band of radiolabeled DNA, said band being indicative of the presence of msDNA in the bacterium.
